(19) United States
(12) Patent Application Publication
(10) Pub. No.: US 2009/0061461 A1
(43) Pub. Date: Mar. 5, 2009

(54) Phosphorylated or Non-Phosphorylated MPR as Diagnostic Marker or Therapeutic Target

(76) Inventors: Michael Cahill, Loerzweiler (DE); Andre Schrattenholz, Mainz (DE); Raffael Kurek, Dieburg (DE); Diethelm Wallwiener, Tubingen (DE); Hans Neubauer, Tubingen (DE); Susan Clare, Chicago, IL (US)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 11/992,699

(22) PCT Filed: Sep. 26, 2006
(86) PCT No.: PCT/EP2006/009351

§ 371 Date: Mar. 26, 2008

(30) Foreign Application Priority Data
Sep. 26, 2005 (EP) 05020917.0
Nov. 24, 2005 (EP) 05025639.5

(51) Int. Cl. G01N 33/567

(52) U.S. Cl. 435/7.21

(57) ABSTRACT

A method of diagnosing diseases associated with aberrant biological phenotypes including contacting a sample to be tested with at least one isoform of membrane associated progesterone receptor component 1 (mPR) as a diagnostic marker, and determining or estimating the degree of phosphorylation of the membrane associated progresterone receptor component 1 (mPR).

### RELATED APPLICATIONS

This is a §371 of International Application No. PCT/EP2006/009351, with an inter-national filing date of Sep. 26, 2006 (WO 2007/039189 A1, published Apr. 12, 2007), which is based on European Patent Application Nos. 05020917.0, filed Sep. 26, 2005, and 05025639.5, filed Nov. 24, 2005.

### TECHNICAL FIELD

This disclosure relates to the use of at least one isoform of membrane associated progesterone receptor component 1 (mPR), as a diagnostic marker in diagnosis for diseases associated with aberrant biological phenotypes, to an assay, to an assay kit usable for the assay, and to the use of reagents that influence the phosphorylation status of mPR and/or the abundance and/or activity of other proteins for diagnosis and/or therapy of diseases associated with aberrant biological phenotypes.

### BACKGROUND

Phosphorylation and dephosphorylation of a protein is one of the fundamental activating and deactivating processes in biological systems, in particular with respect to cellular signal transduction processes. Disturbances relating the phosphorylation status (degree of phosphorylation) of a protein are often associated with aberrant biological phenotypes, particularly uncontrolled cell proliferation, that may cause serious diseases, especially cancer.

For instance, breast cancer is one of the most common forms of cancer observed in women in the western civilization, in particular, in the United States of America with a predicted number of approximately 215,990 (32%) new cases and with over 40,000 deaths expected in 2005. Consequently there is an increasing demand for a better understanding of molecular events, especially the phosphorylation and dephosphorylation of proteins, underlying cancer and other diseases associated with aberrant biological phenotypes to develop improved diagnostic and therapeutic strategies.

Thus, it could be advantageous to provide a protein that is dependent in vivo on phosphorylation and/or dephosphorylation as a diagnostic and/or therapeutic marker, reagents to influence the protein and an appropriate assay allowing for diagnosis and/or therapy of diseases that are related to aberrant biological phenotypes.

### SUMMARY

We provide a method of diagnosing diseases associated with aberrant biological phenotypes including determining or estimating the degree of phosphorylation of at least one isoform of membrane associated progesterone receptor component 1 (mPR) in a sample to be tested.

We also provide a method of diagnosing diseases associated with aberrant biological phenotypes including determining or estimating the degree of phosphorylation of at least one isoform of membrane associated progesterone receptor component 2 (PGRMC2) in a sample to be tested.

We further provide an assay kit that diagnoses and/or treats diseases associated with aberrant biological phenotypes including at least one isoform of membrane associated progesterone receptor component 1 (mPR).

We still further provide a method of treating diseases associated with aberrant biological phenotypes including administering a therapeutically effective amount of at least one reagent that influences the degree of phosphorylation of at least one isoform of membrane associated progesterone receptor component 1 (mPR) to a mammal.

### BRIEF DESCRIPTION OF THE TABLES AND DRAWINGS

The tables and figures relate to the following.

Table 1: pooling design for ER⁺ vs ER⁻ cryogenic whole tumor sections

Individual tumors are designated by their tumor bank T registration numbers. Experimental, clinical and histopathological parameters are listed. Eight ER⁺ and eight ER⁻ tumors are grouped into four pools of two tumors each as indicated. Clinical data comprise: tumor status ranging from pT1 (tumor 2 cm or smaller in greatest dimension) to pT3 (tumor >5 cm); Lymph node status from pN0 (no regional lymphnode metastasis) to pN3 (metastasis to ipsilateral internal mammary lymph nodes(s)) and pNx (regional lymph node cannot be assessed); tumor grade from 2 (moderately differentiated) to 3 (poorly differentiated); Histo-pathological data for ER and PR (0: undetectable, 1-3: weakly positive, 4-7: moderately positive, 8-12: highly positive); and HER2/neu-status (0=negative, positive +1 to +3). Ages for each patient are given in years.

Table 2: protein spots that contained multiple identifications of individual proteins as gene products

The protein name and number of spots are indicated in the column headings. Approximate estimates for the experimentally observed isoelectric point (PI) and molecular weight (MW) are given for each spot, as are Genbank accession numbers and PMF scores, the nomenclature conventions for which follow FIG. 3.

Table 3:

Protein motifs contained in mPR (SwissProt entry O00264). 1. The position within the mPR sequence of the amino acid at the center of the predicted motif is given in the column. 2. The amino acid from column 1 shown in the context of the flanking amino acids that belong to the sequence motif. 3. The type of motif predicted to be present in columns 1 and 2. “Acidophilic S/T Kinase”=acidophilic type serine/threonine kinase. “SH3”=Src homology 3 domain, “Kinase binding”=predicted binding site for a protein kinase. “Tyr-Kinase”=consensus tyrosine kinase phosphorylation site. “SH2”=Src Homology 2 domain, Column 4 gives the specific type of consensus motif from column 3.

Table 4:

Mutations introduced into specific codons of the PGRMC1 ORF in plasmid pcDNA3_MPR_{—}3HA.

Table 5:

Cell count from stable transfection experiments after 2 week selection, with corresponding graphical representation.

FIG. 1: 54 cm differential ProteoTope® analysis

The panels show actual images from an inverse replicate labelled ProteoTope® experiment for one sample pair. (A) Analysis of pooled sample ER⁺¹ (ERpos1) from Table 1 labelled with 1-125, differentially compared with pooled sample ER⁻¹ (ERneg1) labelled with I-131. The lower panels show the signal detected for each isotope, depicted in false spectral color. The signals for each isotope have been normalized against each other for total relative intensity in the upper dual channel images, where the signal for I-125 is blue, the signal for I-131 is orange, and equal amounts of both signals produces grey or black signal. Two pure sources each of I-131 and I-125, as well as a 50% mixture of both isotopes, are measured on round 2 mm pieces of filter paper placed next to each gel as imaging controls. Cross talk between the signals from each isotope is <1%. The pH ranges of the 18 cm IPGs used for serial IEF are indicated above the panels, and the radioactive iodine isotope signals depicted in each panel are indicated on the right. In this experiment all iodination reactions were performed on 60 μg protein. In the examples shown, the I-125 is signal is systematically stronger in all gels (compare lower panels for individual isotopes). (B) The top panels show the inverse replicate experiment of A, where sample ER⁺¹ is labelled with I-131, and sample ER⁻¹ is labelled with I-125. The bottom panel shows an enlarged portion of a gel image, as indicated. Similar gels were produced for all corresponding differential analyses depicted in Table 1.

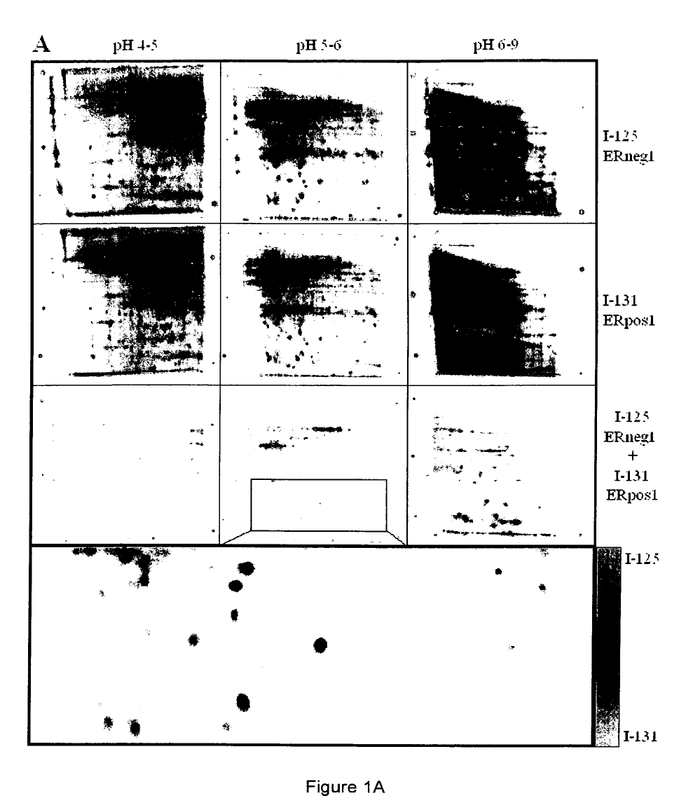

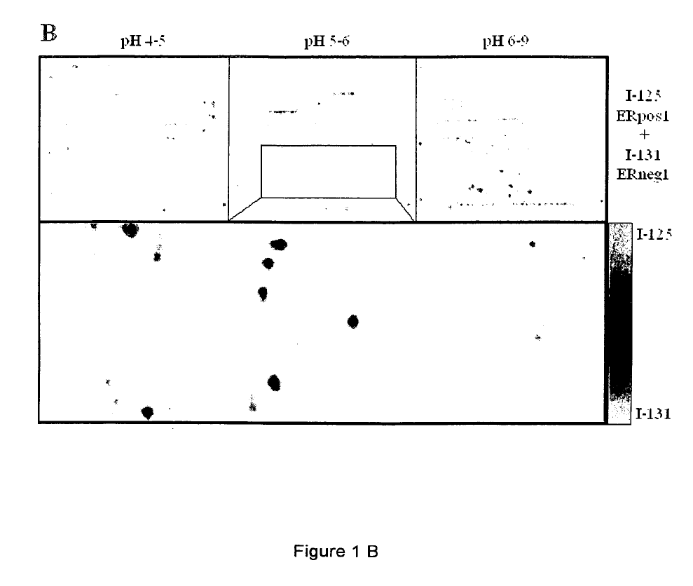

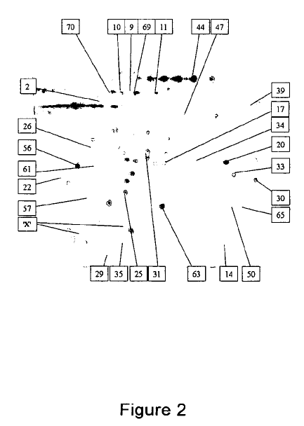

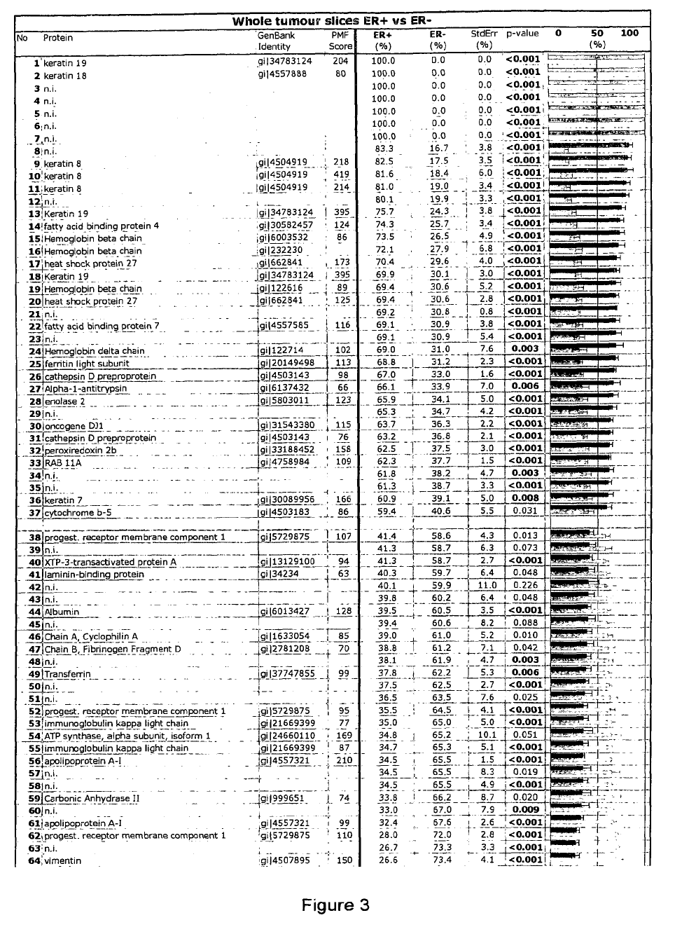

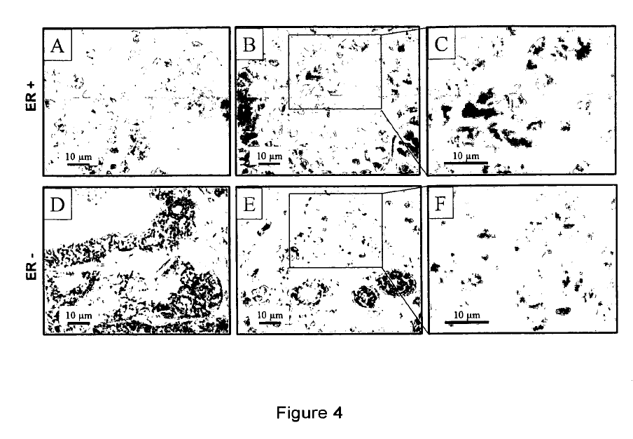

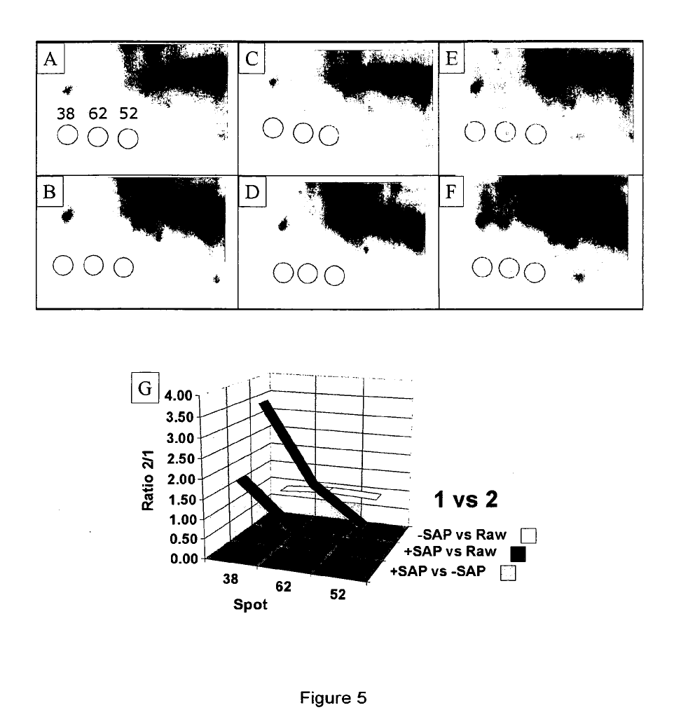

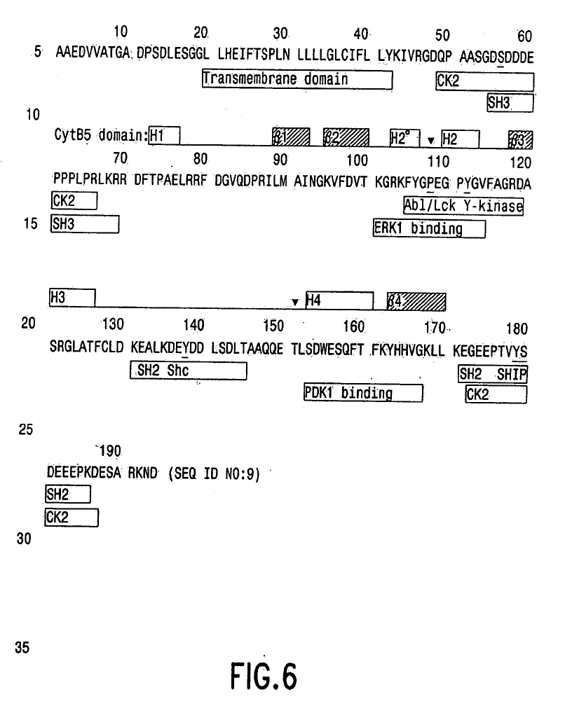

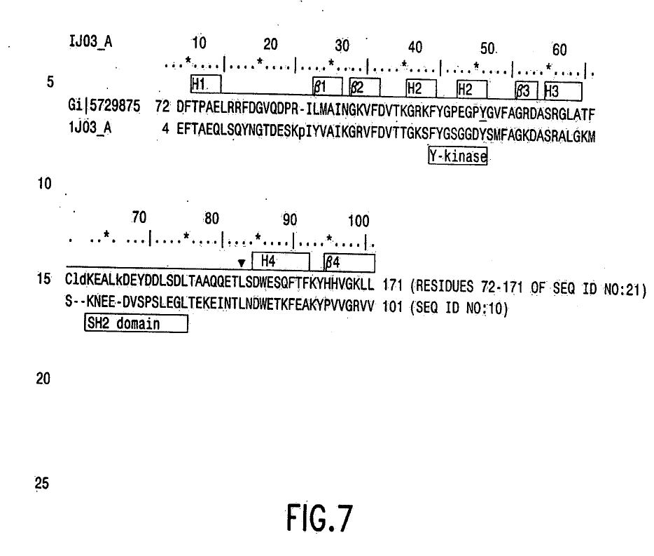

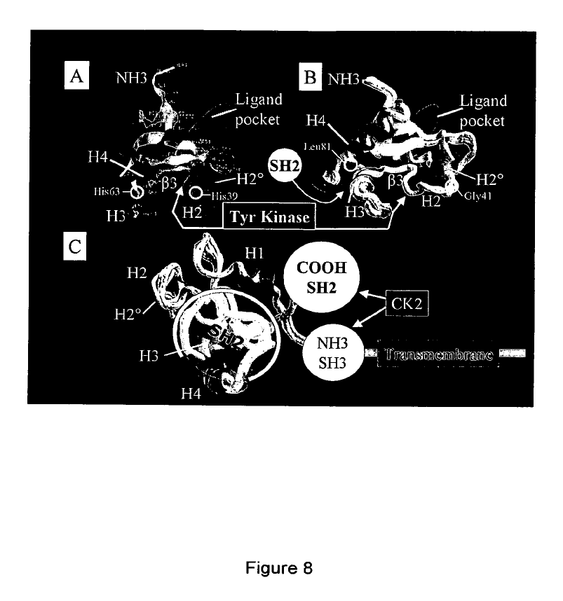

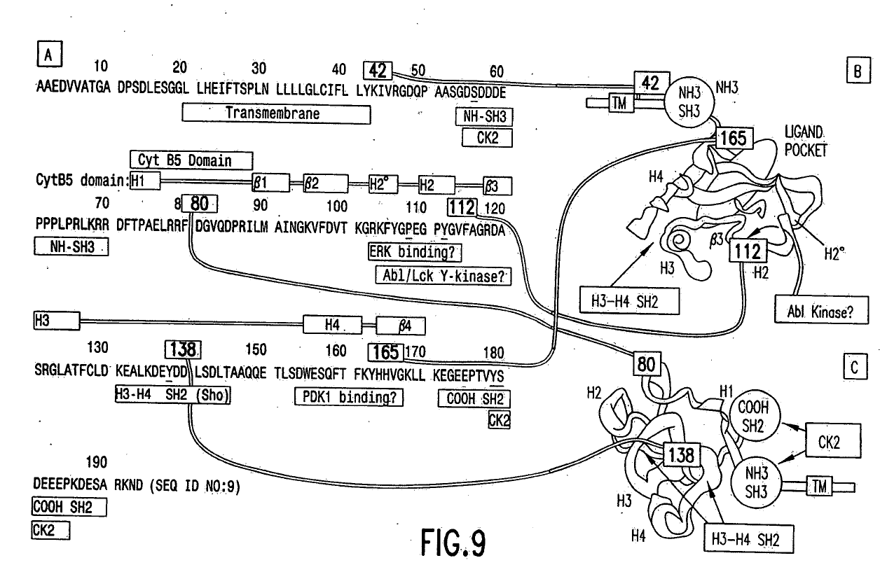

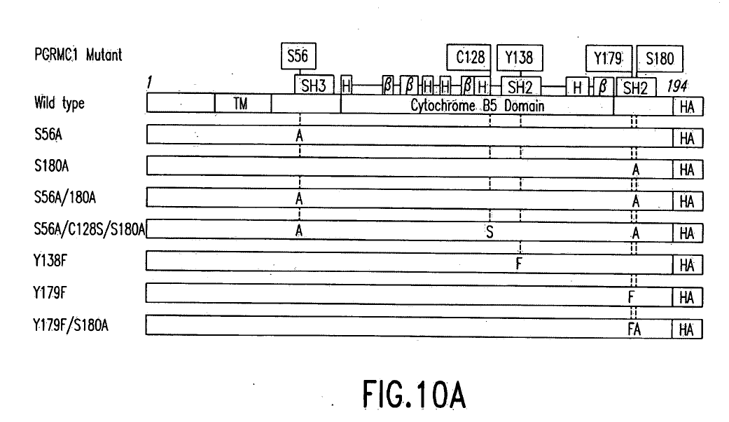

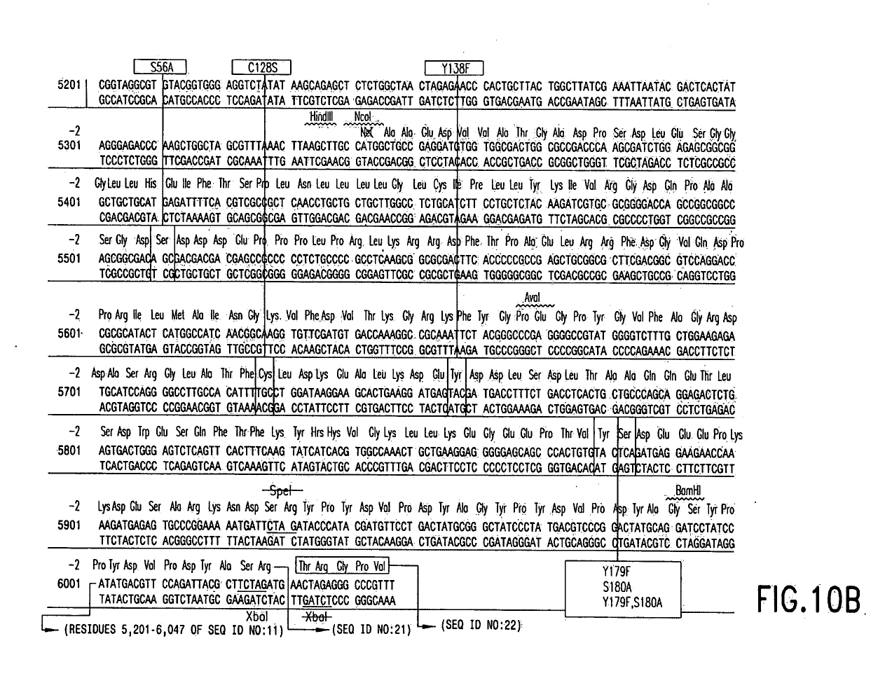

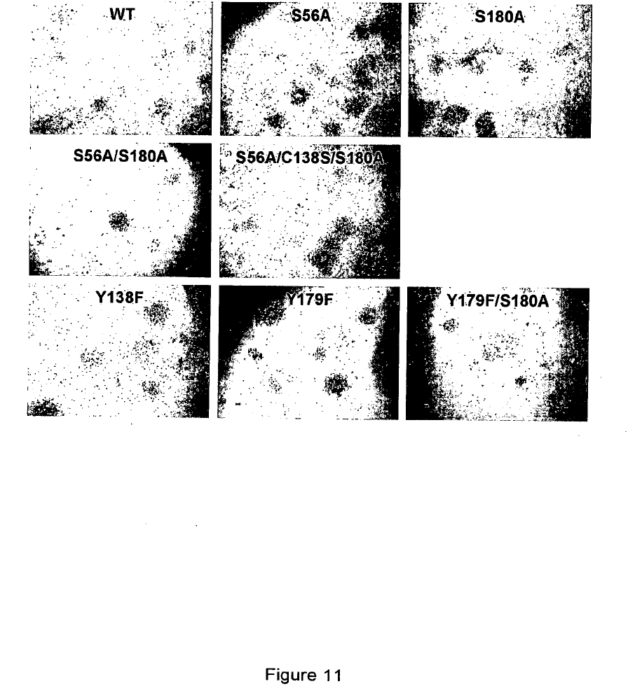

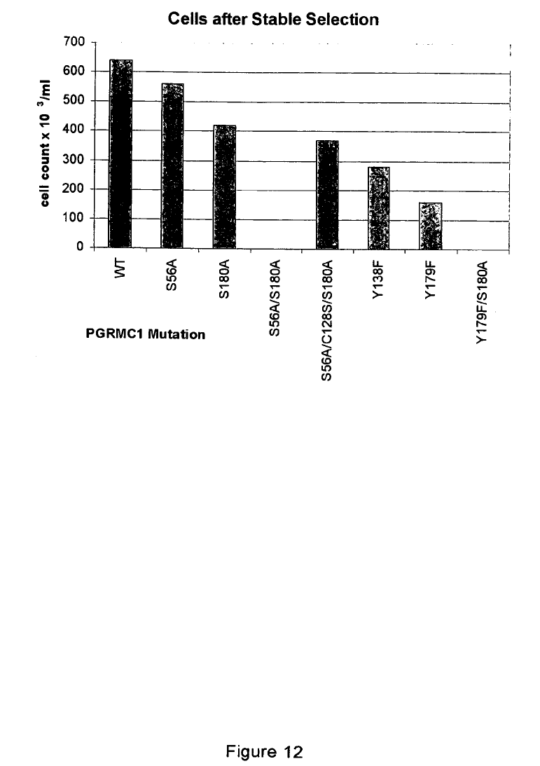

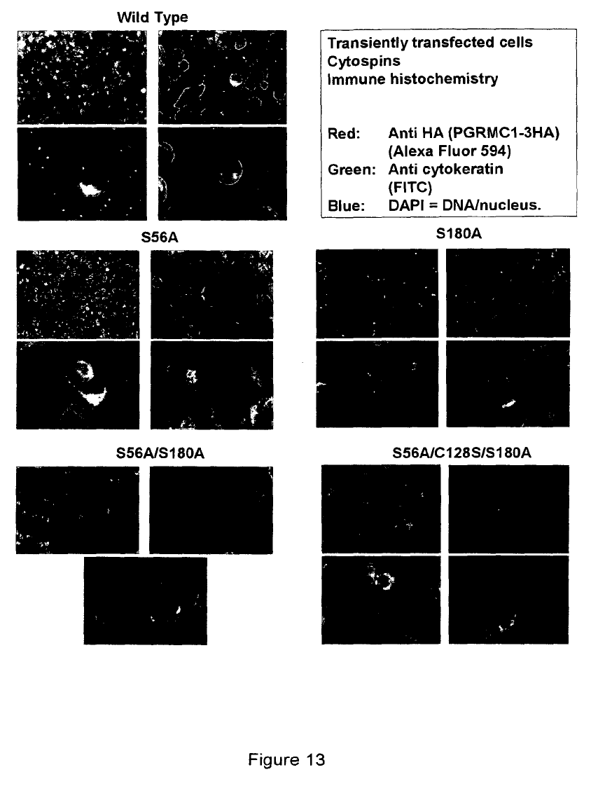

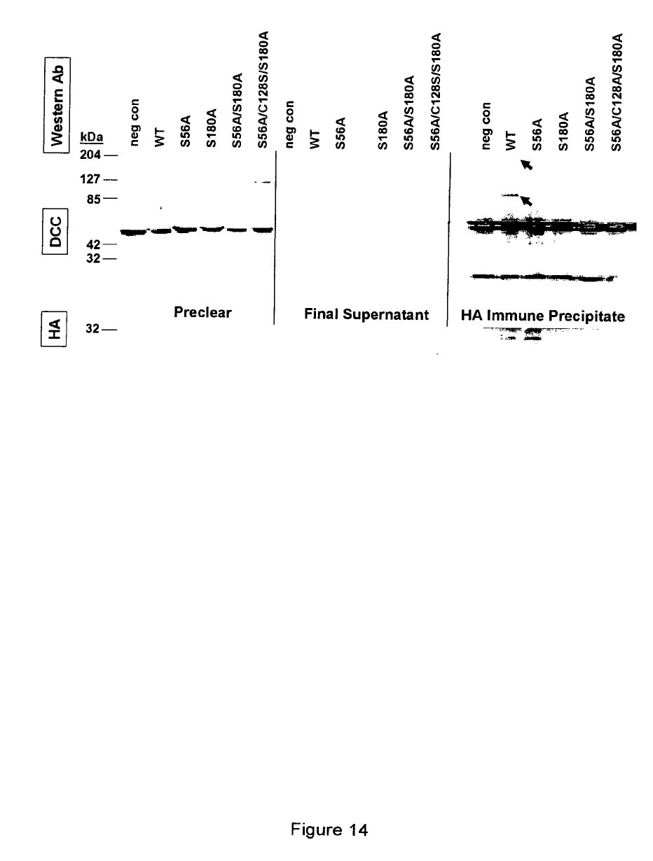

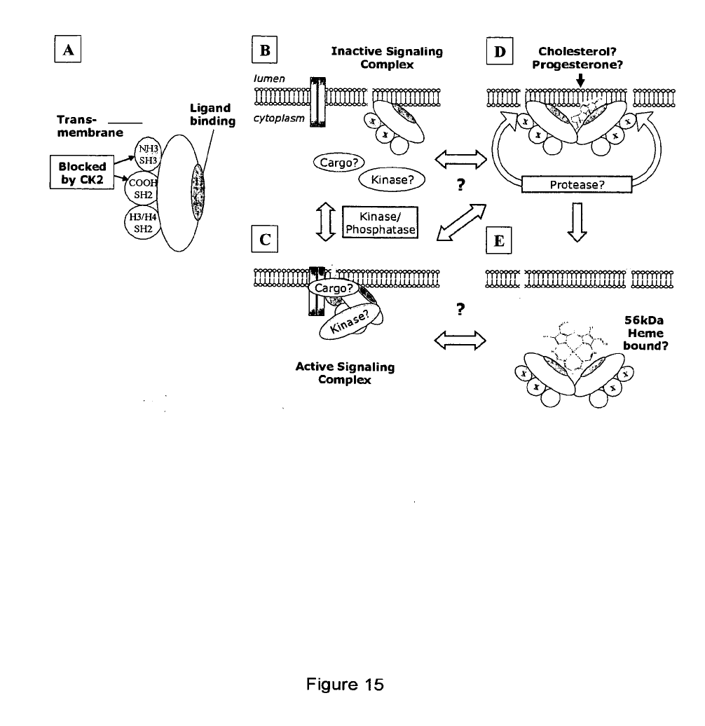

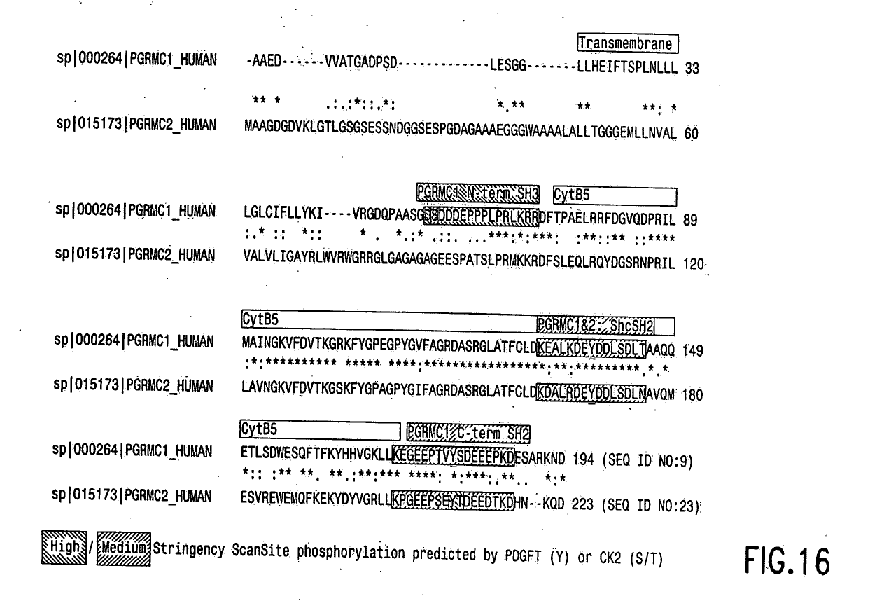

FIG. 2: Typical example of a synthetic average composite gel of the pH 5-6 analysis, showing spots matched across all gels in the study in this pH range from FIG. 1

The average ER⁺ signal is indicated as blue, the average ER signal is indicated as orange, and equal intensities of both signals give grey or black pixels. Spot numbers correspond to FIG. 3. Some orange or blue spots that are not numbered (e.g., those labelled ‘X’) were not visible on preparative silver stained tracer gels, and were omitted from the analysis. This image was generated with the GREG software. Labels were added manually.

FIG. 3: Protein spot quantification and identifications for breast cancer samples (whole tumor slices) comparing ER positive and ER negative samples

n.i.=not identified. Genbank Identities are from the NCBI data base version of Apr. 4, 2004. MALDI-TOF peptide mass fingerprinting (PMF) scores are from MASCOT. The average spot fraction for ER⁺ and ER⁻ are given as percent of the normalized total spot volume for each spot (=(ER⁺×100%)/(ER⁺+ER⁻)) across all patient pools based on two color ProteoTope® analysis for the indicated most significant protein spots. These values were obtained using a least square fit for a model based on all replicates and attributing pool variability as a random effect. The t-test p-value for this model is also given. P-values <0.01 are bold, and p-values <0.001 are designated as such. The bars at the right depict average percent abundance of each protein across the ER⁺ (dark blue) and ER⁻ (light orange) pools as indicated above the column with bars (0%-50%-100%). Error bars show standard error of means. Protein spots between numbers 37 and 38 (indicated by a grey field) are not presented, having failed to meet selection criteria of either abundance difference ratio of 1.5 or significance at the 5% level.

FIG. 4: mPR immune histochemistry in ER⁺ and ER⁻ tumors

The rabbit polyclonal anti-mPR-specific signal (green) is associated with diffuse cytoplasmic staining in ER⁺ tumors (A-C), whereas anti-mPR signal exhibits increased localized concentration to specific extra-nuclear sub-cellular locations in ER⁻ cells (D-F). The dark purple color is hematoxylin counter-staining of nuclear chromatin. The 10 μm scale bar is shown in each panel. A and B show the mPR staining pattern of two different tumors, while C shows an enlargement of the framed region from B, as indicated. The same relationship applies to D, E and F. The rabbit polyclonal antiserum was a gift of F. Lösel (University fo Heidelberg).

FIG. 5: Differential quantification of phosphatise treated and control samples

(A-F) Inverse replicate ProteoTope® images of the gel region containing three spots of mPR: spots 38, 52, and 62 from FIG. 3. Image conventions follow FIG. 1. (A) The phosphatase treated sample (+SAP) is labelled with I-125 (blue color), and the mock incubation control (−SAP) is labelled with 1-131 (orange color). Spot numbers are indicated, and are applicable to all panels. (B) The inverse replicate experiment to A. (C) I-125 labelled +SAP is analyzed against 1-131 labelled untreated raw control sample (raw). (D) The inverse replicate experiment to C. (E) I-125 labelled −SAP is analyzed against 1-131 labelled raw control. (F) The inverse replicate experiment to E. (G) Quantification of the differential ratio of signal intensities from sample 2/sample 1 for each of the spots from the gels shown in A-F. The identity of sample 1 and sample 2 for each comparison are shown at the right hand side of the panel, with corresponding color coding. The ratio of signal for control and treated samples increases in a phosphatase-dependent manner, consistent with spots 38 and 62 representing phosphorylated isoforms of spot 52.

FIG. 6: Position of structural motifs conserved for the mPR Cyt-b₅ domain

Amino acids of the cyt-b₅ (cytochrome b₅) domain of mPR numbered according to SwissProt Accession O00264 (SEQ ID NO:10) are boxed and shaded. Helices (H1-H4) and beta strands (β1-β4) are as published (Mifsud and Bateman, 2002), except helix H2°, which was added by the authors according to the crystal structure of bovine cyt-b₅. Triangles above G107 and L152 represent positions in the structure where corresponding histidine residues interact with the ligand heme group in the structure of cyt-b₅. Amino acids predicted to be phosphorylated at consensus kinase sites are underlined. In addition to the motif predictions from Table 3, the predicted transmembrane domain from SwissProt is indicated between amino acids 20-42. FIG. 6 shows the sequence of membrane associated progesterone receptor component 1 (mPR) (SEQ ID NO:9).

FIG: 7:

Sequence alignment of the cytochrome b₅ domains of mPR (gi|5729875) and the Arabidopsis Putative Steroid Binding Protein 1J03_A. Amino acid numbering is that of 1J03_A. Conserved amino acids are in bold print. Other notation follows the convention of FIG. 6 (the mPR sequences designated by SwissProt Accession O00264, and NCBI GeneBank Identifier gi|5729875 represent the same protein, but differ by the presence or absence of the N-terminal initiator methionine).

FIG. 8: Structural modelling of the cytochrome b₅ domain of mPR, and flanking motifs, indicating structural domains following FIG. 6

Structures were manipulated using the RasMol program. (A) The crystal structure of bovine cyt-b₅ (PDB Accession 1CYO). The ligand binding pocket is indicated, and the orthologous position of the predicted tyrosine kinase phosphate acceptor from mPR (FIG. 6) is indicated in A and B. The position of ligand-interacting His39 and His63 are also indicated, as are the corresponding Gly41 and Leu81 in B. (B) The NMR structure of Arabidopsis “Putative Steroid-Binding Protein” (PDB Accession 1J03_A) shown looking down onto the ligand binding pocket in similar orientation to structure in A. (C) The structure from B is rotated to view the opposite surface, showing the inferred adjacent locations of the src homology domain structural motifs predicted for mPR. The position where the predicted SH3 domain at the N-terminal, and SH2 domain at the C-terminal of the superposed cytochrome b₅ domain of mPR are schematically indicated, as are the helix-3/helix-4 SH2 domain and the N-terminal transmembrane region.

FIG. 9: mPR modelled using the Arabidopsis 1J03_A NMR structure

(A) The amino acid sequence of mPR (SwissProt O00264), showing predicted functional motifs from Table 1 below the sequence. The cytochrome b5 domain is indicated above the sequence, with positions of helices and beta sheets according to FIG. 7. The position of tyrosines of the putative ITAM/YXX(Φ) motifs are shown in boxes above the amino acid sequence (boxes 42, 80, 112, 138 and 165). (B) and (C) show the structure of mPR as modelled with the Arabidopsis 1J03_A coordinates (which were depicted with RasMol), showing the positions of the boxes from (A) (B) view from the side of the ligand binding pocket. (C) View from the side of the cytochrome b5 domain on the other side to the ligand binding pocket. The position of mPR features which are not present in the 1J03_A structure are schematically portrayed by circles to correspond with (A).

FIG. 10: Amino acid mutations introduced into PGRMC1

A. Schematic representation of the mPR ORF, amino acids 1-194 plus three C-terminal 3xhemaglutinin (HA) tags in plasmid pcDNA3_MPR_{—}3HA (Wild type). The transmembrane domain (TM) and Cytochrome B5 domain are indicated. Amino acid numbering is according to human mPR Uniprot O00264, which does not include the initiator methionine (as deleted in the figure). Uniprot sequence Q6IB11 corresponds to the same sequence including the initiator methionine, whereby the mutated human mPR amino acids would be numbered as Ser57, Cys129, Tyr139, Tyr180, Ser181, etc., as hereby disclosed. B. The nucleotide sequence of the section of plasmid pcDNA3_MPR_{—}3HA encoding the mPR ORF with C-terminal HA tags, showing the locations of mutations that were constructed by site-directed mutagenesis. The codons used to generate the amino acid mutations are shown in the Table 4.

FIG. 11: Colony formation of transfected MCF-7 cells after 2 weeks of selection

2×10⁶ cells were transfected with the indicated plasmids under identical conditions and plated. The representative panels show colonies after 2-weeks of selection.

FIG. 12: Graphical representation of the stable selection of PGRMC1 mutants as shown in Table 5

FIG. 13: Immune histochemical subcellular localization of transiently transfected m PR (red) and mutants thereof as indicated

Whereas the wild type protein exhibits colocalization with cytokeratin, none of the mutant proteins do, indicating different subcellular interaction partners and localizations for PGRMC1 variants used in this experiment. The meaning of colors is indicated in the figure. Yellow shows colocalization of red and green. No physical interaction between mPR and keratin is intended to be shown beyond general colocalization.

FIG. 14: Immune precipitation of DCC with mPR

Immune precipitation of DCC with mPR depends upon serine 56 and serine 180. Bands reacting specifically with DCC in the mPR wild-type immunoprecipitate are indicated by arrows. Cells were transfected with each of the indicated mPR expression plasmids or with the empty plasmid vector control (neg con). The Western blot of the upper panels was developed after incubation with anti-DCC AF5 antibody. The lower band shows the same membrane after stripping and incubating with the anti-HA antibody which was used to immuno-purify the HA-tagged mPR proteins. The most prominent dark bands in the upper panels represent primarily the heavy and light chains of the anti-HA antibody which was used for immunoprecipitation.

FIG. 15: Hypothetical model for mPR function as an adapter molecule in signalling

(A) Schematic representation of putative mPR functional modules, based upon the FIG. 6 to FIG. 9. (B) According to the model, when mPR is phosphorylated by CK2 at S56 and S180 the putative SH3 and SH2 target sequences are unavailable to interact with SH3- and SH2-domain-containing proteins, describing the possible situation in ER⁺ tumors. One or more CK2 sites are phosphorylated (“x”), inactivating the interaction with other proteins (e.g., membrane receptors and “Cargo?”) through the respective SH3 and/or SH2 domains N- and C-terminal to the Cyt-b₅ domain. One or more kinases may be bound to mPR, such as ERK1 or PDK1 to their respective predicted sites, or a tyrosine-phosphorylated signal transduction-effecting molecule such as a tyrosine kinase or phosphatase to probably the Helix3-Helix4 SH2 domain. (C) The situation in ER⁻ tumors. The CK2 site phosphorylation state is reduced, permitting interaction with cargo proteins and signaling receptors to form active signal transduction complexes. Possibly, ligand binding could affect the situation in C. This association is a further part. (D) Possible cholesterol and/or steroid binding may require dimerisation of mPR from a ‘28 kDa’ monomer to a ‘56 kDa’ dimer. (E) Protease action, such as by the S2P protease which cleaves SREBP, may release a ‘56 kDa’ dimer to the cytoplasm, where it can bind heme. These speculative scenarios are intended to be functionally illustrative, not mutually exclusive. Translocation(s) between sub-cellular locations may be involved in change between functional scenarios (straight double arrows).

FIG. 16: Alignment mPR vs PGRMC2

The positions of transmembrane domain (blue), cytochrome b5 domain (white box) and the putative SH3 and SH2 target sequences are shown for mPR, as well as corresponding putative functional features from PGRMC2 where they are present. The location of putative phosphate acceptor sites predicted by MotifScan under high (red) or medium (brown) stringency settings are also indicated for both proteins. The putative tyrosine phosphate acceptors for SH2 target sequences are in underlined bold font.

Alignment was performed with the Expasy sequence aligner function (http://www.expasy.org/cgi-bin/aligner?seq=O15173&seq=O00264) which was accessed from the Expasy BLAST results page.

### DETAILED DESCRIPTION

A phosphorylated and/or non-phosphorylated membrane associated progesterone receptor component 1 (mPR), in particular, at least one isoform thereof, is used as a diagnostic marker and/or therapeutic target for diseases that are associated with aberrant biological phenotypes.

At least one isoform of a membrane associated progesterone receptor component 1 (mPR) is used, as a diagnostic marker in diagnosis for diseases associated with aberrant biological phenotypes, wherein the phosphorylation status, i.e., the degree of phosphorylation of membrane associated progesterone receptor component 1 (mPR) is determined and/or estimated.

The term “phosphorylation status” as used herein comprises the absolute or relative degree of phosphorylation of proteins and/or reagents.

At least one isoform of a membrane associated progesterone receptor component 2 (PGRMC2) is used, as a diagnostic marker in diagnosis of diseases associated with aberrant biological phenotypes, wherein the phosphorylation status, i.e., the degree of phosphorylation of a membrane associated progesterone receptor component 2 (PGRMC2) is determined.

No discussion of mPR can be complete without consideration of its close relative, PGRMC2. This protein is also known in literature as VemaB and hIZA2. Although the function of PGRMC2 is yet unknown, it shows certain similarities to mPR. A sequence alignment, considering the position of putative functional domains is shown in FIG. 16. The overall topology of both proteins is expectantly similar since they are derived from one pre-vertebrate ancestral gene. The amino acids N-terminal to the transmembrane domain are remarkably different between the proteins. These could mediate contact with different interaction partners in the lumen of sub-cellular organelles, or on the surface of the cytoplasmic membrane. The putative SH3 target sequence between the transmembrane domain and the cytochrome b5 domain of mPR, with its consensus acidophilic kinase site (nominally CK2 in this discussion), is totally absent from PGRMC2, suggesting another avenue by which these proteins could perform disparate roles. The cytochrome b5 domain itself is highly conserved, including the region for putative ERK binding and Lck/Abl tyrosine kinase binding, and the putative SH2 target sequence between helices H3 and H4. The C-terminal putative SH2 target sequence is also present in both proteins. The putative phosphate accepting tyrosine of both proteins is flanked C-terminally by the phosphate-acceptor of a consensus CK2 site in both instance, serine in mPR and threonine in PGRMC2. These sites are predicted under high stringency settings to be phosphate-acceptors for PDGFR beta and for CK2 kinases respectively by ScanSite Motifscan. PGRMC2 possesses additional potential CK2 sites in this region, that are predicted using medium stringency Motifscan settings. Mutational analyses will be required to determine whether these homologous putative SH2 target regions exert overlapping or distinct functions.

According to a preferred example, the phosphorylation status may be determined and/or estimated by analysis of proteins that are involved, in particular, differentially involved in protein interaction or multi-protein complexes with either phosphorylated or non-phosphorylated membrane associated progesterone receptor component 1 (mPR).

According to a preferred example, the phosphorylation status may be determined and/or estimated by analysis of proteins that are involved, in particular, differentially involved in protein interaction or multi-protein complexes with either phosphorylated or non-phosphorylated membrane associated progesterone receptor component 2 (PGRMC2).

According to a preferred example, the phosphorylation status of the mPR may be determined by means of affinity reagents, in particular, by means of antibodies.

According to a preferred example, the phosphorylation status of the PGRMC2 may be determined by means of affinity reagents, in particular, by means of antibodies.

As affinity reagents that can be used, any reagent or method that is known or will be known to one skilled in the art. For example, antibodies, aptamers, RNA display, phage display or combinations thereof may be used.

According to a preferred example, the phosphorylation status of the mPR is determined by means of incorporating radioactive atoms into the phosphate group, or derivatizing the phosphate in other ways (such as elimination and Michael-addition) such that the presence of original phosphate groups can be detected by methods and by reagents, including by way of example, but not limited to, fluorescence or surface Plasmon resonance.

According to a preferred example, mPR, in particular, at least one isoform thereof, is at least partially phosphorylated when used as diagnostic marker and/or therapeutic target for diseases that are associated with aberrant biological phenotypes.

“Membrane associated progesterone receptor component 1” (mPR, also known as Hpr6.6, or progesterone membrane receptor component 1/PGC1/PGRMC1/PGRC1) as used herein, comprises the entire mPR protein or any partial sequence thereof, if appropriate synthetically manufactured, in particular by means of genetic engineering, wherein the partial sequence reveals the activity of mPR. Additionally, protein fragments remaining after proteolytic cleavage of the transmembrane domain from the rest of mPR and/or proteins being partially homologous thereto, and their use as medicines, as above are explicitly covered by the term “Membrane associated progesterone receptor component 1 (mPR).” In particular, the term “Membrane associated progesterone receptor component 1 (mPR)” shall comprise homologous proteins from various species like “predicted 25 kDa protein upregulated by dioxin” (25-Dx), “membrane progesterone receptor” (for example derived from swine liver membrane), “heme progesterone receptor 6.6” (Hpr6.6, simply denoted as Hpr6 or human membrane progesterone receptor (hmPR)), “ventral midline antigen” (VEMA or VemaA), “CAudalROstral 2” (CARO 2), from rat derived forms like ratp28 (195 amino acid residues) or HC5 (75 amino acid residues), and from rat derived “inner zone antigen” (IZA). Other yet unknown proteins of the mPR-family shall also be comprised by the term mPR. Because of the high degree of homology between mPR/PGRMC1 and the related family member PGRMC2 in the C-terminal region of the native protein, including the phosphorylation positions, PGRMC2 is claimed.

We note that another protein, termed membrane progestin receptor, has also been denoted as mPR in the literature. This different gene product is a G-protein coupled seven membrane domain progestin receptor found from fish to mammals that conveys non-genomic effects of progesterone, and is otherwise not at all related to the mPR claimed. There are currently eleven mammalian members belonging to this separate gene family, which has been named the PAQR family, after two of the initially described ligands (progestin and adipoQ receptors).

The term “aberrant biological phenotypes” as used herein comprises all forms of aberrant biological in vivo manifestations, for instance uncontrolled cell proliferation.

“Activity” of proteins as used herein, comprises the enzymatic activity, binding affinity and/or posttranslational activity, in particular phosphorylation.

The term “abundance” as used herein is equivalent to the expression level of proteins, in particular of mPR, being detectable with prior art methods, in particular with the two dimensional ProteoTope® analysis method.

The membrane associated progesterone receptor component 1 (mPR) is not related to the classical or cytoplasmic progesterone receptor (cPR). The mPR has a transmembrane domain N-terminally to a cytochrome b₅ domain that may interact with heme groups, and is probably involved in steroid binding, in particular it has been suggested to be involved in progesterone binding although no physical binding data have been published. Such membrane-associated progesterone receptors (MAPR) represent a family of gene products found in various organisms, and are thought to mediate a number of rapid cellular effects not involving changes in gene expression (Losel, R., Christ, M., Eisen, C., Falkenstein, E., Feuring, M., Meyer, C., Schultz, A. and Wehling, M. (2003) Novel membrane-intrinsic receptors for progesterone and aldosterone. In Watson, C. S. (ed.) The identities of membrane steroid receptors. Kluwer Academic Publishers, Boston, pp. 125-129.). Obviously, they also have the potential to influence gene expression in addition to rapid genome-independent effects. The Hpr6.6/mPR reportedly mediate cell death after oxidative damage through a non apoptotic pathway (Hand R. A., Craven R. J., 2003, Hpr6.6 Protein Mediates Cell Death From Oxidative Damage in MCF-7 Human Breast Cancer Cells, J. Cell. Biochem., 90: 534-547). Other results suggest the opposite wherein mPR/Hpr6 increases cell survival following chemotherapy (Crudden G., Chitti, R. E., Craven R. J., 2005, Hpr6 (heme-1 domain protein) regulates the susceptibility of cancer cells to chemotherapeutic drugs, JPET, 1-23).

We provide evidence of three isoforms of the membrane associated progesterone receptor component 1 (mPR) that is described below. As a result, it is in particular preferred to use any combination of these isoforms as diagnostic markers and/or therapeutic targets for diseases that are associated with aberrant biological phenotypes.

In a particular example, the diseases, in particular, subgroups thereof, comprise cancer, neurodegenerative diseases, infertility, inflammatory, respiratory and/or pulmonary diseases, wherein cancer, especially breast cancer or prostate cancer, is in particular preferred.

According to an especially preferred example, phosphorylated and/or non-phosphorylated membrane associated progesterone receptor component 1 (mPR), in particular, at least one isoform thereof, may be used as a diagnostic marker and/or therapeutic target for subgroups of diseases associated with aberrant biological phenotypes, particularly of cancer, preferably of breast cancer.

For instance, such subgroups can relate to the abundance of at least one protein, in particular of at least one receptor protein, preferably of estrogen receptor. The estrogen receptor (ER) comprises two types of specific nuclear receptors that are known as estrogen receptor a (ERα) and estrogen receptor β (ERβ). Molecular analysis has proven that ERα, like other nuclear receptors, consists of separable domains responsible for DNA binding (DNA binding domain DBD), hormone binding (hormone binding domain HBD) and transcriptional activation domain. The N-terminal activation function (AF-1) of the purified receptor is constitutively active, whereas the activation function located within the C-terminal part (AF-2) requires hormone for its activity.

ERα is found in 50-80% of breast tumors and ERα status is essential in making decisions about endocrine therapy with anti-estrogens, which are competitive inhibitors of endogenous estrogens and inhibit mitogenic activity of estrogens in breast cancer. On a molecular basis, they trigger inactive conformation of the ERα, which is then unable to activate transcription (Shiau A K, Barstad D, Loria P M, Cheng L, Kushner P J, Agard D A, Greene G L. 1998. The structural basis of estrogen receptor/coactivator recognition and the antagonism of this interaction by tamoxifen. Cell. 95: 927-37.).

Clinically, a positive ER status (ER⁺, tumor cells showing abundance of ER) correlates with favorable prognostic features including a lower rate of cell proliferation and histologic evidence of tumor differentiation. In contrast to that, a negative ER status (ER⁻ tumor cells showing no or at least a decreased-abundance of ER) corresponds to substantially poorer disease-free and overall survival probability of the patient. ER status is also prognostic for the site of gross metastatic spread. Besides, tumors with high abundance of estrogen receptor (ER⁺ tumors) are more likely to initially manifest clinically apparent metastasis in bone, soft tissue or the reproductive and genital tracks, whereas tumors with low abundance of Estrogen receptor (ER⁻ tumors) more commonly metastasize to the brain and liver. Several studies have correlated ERα expression to lower Matrigel invasiveness and reduced metastatic potential of breast cancer cell lines (Platet N. Prevostel C, Derocq D, Joubert D, Rochefort H, Garcia M. 1998. Breast cancer cell invasiveness: correlation with protein kinase C activity and differential regulation by phorbol ester in estrogen receptor-positive and -negative cells. Int. J. Cancer. 75: 750-6, and Thompson E W, Paik S, Brunner N, Sommers C L, Zugmaier G, Clarke R, Shima T B, Torri J, Donahue S, Lippman M E, Martin G R, Dixon R B. 1992. Association of increased basement membrane invasiveness with absence of estrogen receptor and expression of vimentin in human breast cancer cell lines. J. Cell. Physiol. 150: 534-544.).

Moreover, when ERα-positive cells are implanted in nude mice, tumors appear only in the presence of estrogens and are poorly metastatic as compared to those developed from ERα-negative (ERα⁻) breast cancer cell lines (Price J E, Polyzos A, Zhang R D, Daniels L M. 1990. Tumourigenicity and metastasis of human breast carcinoma cell lines in nude mice. Cancer Res. 50: 717-721).

For instance, we found that mPR was significantly more abundant in breast cancer cells showing a negative ER status (ER⁻) compared to breast cancer cells showing a positive ER status (ER⁺). Besides, we provide evidence for different degrees of phosphorylation of mPR in breast cancer cells differing in the ER status. The alignment of the phosphorylation status of mPR to subgroups of diseases that are associated with aberrant biological phenotypes, in particular cancer, preferably breast cancer, is advantageous with respect to choice or effectiveness of therapeutic treatments.

For instance, with regard to patients suffering from breast cancer exhibiting a positive ER status tamoxifen is effective in approximately 50% of the cases (Fisher B, Jeong J, Dignam J, Anderson S, Mamounas E, Wickerham D L, and Wolmark N. 2001. Findings from recent National Surgical Adjuvant Breast and Bowel Project adjuvant studies in stage I breast cancer. J Natl Cancer Inst Monogr 30: 62-66.).

In addition, we were the first to detect a wound response signature in breast cancer cells showing a negative ER status (ER⁻) by proteomics, and associated this with a decreased phosphorylation of the membrane associated progesterone receptor component 1 (mPR).

In a further example, mPR is derived from mammalian samples, in particular, from human samples.

In a further example, PGRMC2 is derived from mammalian samples, in particular, from human samples.

In a further example, samples are harvested by biopsy and/or surgical extraction.

We use at least one reagent for influencing, in particular, increasing or inhibiting the phosphorylation status, i.e., the degree of phosphorylation of at least one isoform of membrane associated progesterone receptor component 1 (mPR) for diagnosis and/or therapy of diseases related to aberrant biological phenotypes. We show that a reagent capable of influencing the phosphorylation of mPR can be therapeutically effective, which is supported by the examples described herein.

In a preferred example, at least one isoform of phosphorylated and/or non-phosphorylated membrane associated progesterone receptor component 1 (mPR) is used as a diagnostic marker and/or therapeutic target for diseases associated with aberrant biological phenotypes.

In a preferred example, at least one isoform of phosphorylated and/or non-phosphorylated membrane associated progesterone receptor component 2 (PGRMC2) is used as a diagnostic marker and/or therapeutic target for diseases associated with aberrant biological phenotypes.

Furthermore, we use at least one reagent for influencing, in particular, increasing or inhibiting the phosphorylation status (degree of phosphorylation) of membrane associated progesterone receptor component 1 (mPR), in particular, of at least one isoform thereof, for the manufacture of a medicament and/or pharmaceutical composition for diagnosis and/or therapy of diseases associated with aberrant biological phenotypes.

Furthermore, we use at least one reagent for influencing, in particular, increasing or inhibiting the phosphorylation status (degree of phosphorylation) of membrane associated progesterone receptor component 2 (PGRMC2), in particular, of at least one isoform thereof for the manufacture of a medicament and/or pharmaceutical composition for diagnosis and/or therapy of diseases associated with aberrant biological phenotypes.

In some cases, it is desirable that mPR is not completely dephosphorylated. Thus, the reagent decreases the degree of phosphorylation of mPR to a certain extent. In other cases, it may be beneficial to maintain the phosphorylation status of mPR. Therefore, at least one reagent may be used as a diagnostic marker and/or therapeutic target that maintains the phosphorylation status of mPR, in particular, at least one isoform thereof.

Furthermore, we use at least one reagent that is used for diagnosis and/or therapy of diseases associated with aberrant biological phenotypes for influencing, in particular, increasing or inhibiting the interaction of at least one isoform of membrane associated progesterone receptor component 1 (mPR) with other molecules, especially proteins. Preferably, the interaction of at least partially phosphorylated membrane associated progesterone receptor component 1 (mPR) is influenced, in particular, increased or inhibited, by the reagent.

In a particular example, at least one reagent is used that binds to at least partially phosphorylated, preferably completely phosphorylated, mPR and/or PGRMC2 to prevent interaction of Src Homology 2 (SH2) and SH3 target amino acids with other molecules, particularly proteins. With respect to molecular details concerning the structure and mechanistic studies of mPR, it is referred to the following description.

It is in particular advantageous that the reagent is a ligand of mPR that in particular binds to the ligand binding pocket of mPR, or to protein interaction domains of the SH2 and SH3 variety on other proteins which interact with mPR, or with the target sequences for those SH2 and SH3 domains in the mPR protein to prevent interaction of mPR with other molecules, especially proteins, allowing for diagnosis and/or therapy of diseases associated with aberrant biological phenotypes.

Additionally, we use proteins, in particular, isoforms thereof, as diagnostic markers and/or therapeutic targets and/or medicines for diseases associated with aberrant biological phenotypes, wherein the proteins are involved, in particular, differentially involved, in protein interaction or multi-protein complexes with either phosphorylated or non-phosphorylated membrane associated progesterone receptor component 1 (mPR). With respect to further details, particularly with respect to phosphorylated or non-phosphorylated membrane associated progesterone receptor component 1 (mPR), it is referred to the above description.

The interactions can be observed for example in binding of antibodies directed against mPR, in particular in binding of monoclonal antibody C-262 (StressGen, Victoria, BC, Canada) and in coimmunoprecipitation of gamma aminobutyric acid A (GABAA) (Peluso J J, Pappalardo A, 1998. Progesterone mediates its anti-mitogenic and anti-apoptotic actions in rat granulosa cells through a progesterone-binding protein with gamma aminobutyric acidA receptor-like features. Biol Reprod 58: 1131-1137). Digitonin dependant coimmunoprecipitation of mPR and caveolin with antisera to caveolin (Bramley T A, Menzies G S, Rae M T, Scobie G 2002 Non-genomic steroid receptors in the bovine ovary. Domest Anim Endocrinol 23: 3-12) and presence of ITAM motifs (YXX(Φ), where Φ represents an aliphatic amino acid) support the possible function of mPR as an adaptor protein involved in regulating protein interactions involved in membrane trafficking, such as endocytosis, exocytosis, or vesicle biology, as well as associated intracellular signal transduction. The presence of a higher order complex of mPR, in particular at least a dimerized form, was indicated by the reduction of disulfide bridges by dithiothreitol (DTT) (Falkenstein E, Eisen C, Schmieding K, Krautkramer M, Stein C, Losel R, Wehling M 2001 Chemical modification and structural analysis of the progesterone membrane binding protein from porcine liver membranes. Mol Cell Biochem 218: 71-79).

In biotinylation experiments it was shown that mPR was present in an immune pellet precipitated by an antibody directed against a protein denoted as “plasminogen activator inhibitor RNA-binding protein 1” (PAIRBP1) from progesterone-responsive ovarian epithelial cells, and that both coprecipitated proteins in this complex were biotinylated in non-permeabilized cells, indicating that they were present on the outer cell surface (Peluso J J, Pappalardo A, Losel R, Wehling M, 2005, Expression and function of PAIRBP1 within gonadotropin-primed immature rat ovaries: PAIRBP1 regulation of granulosa and luteal cell viability. Biol Reprod 73: 261-270; and Peluso J J, Pappalardo A, Losel R, Wehling M, 2006, Progesterone membrane receptor component 1 expression in the immature rat ovary and its role in mediating progesterone's antiapoptotic action. Endocrinology 147: 3133-3140). Besides, it was demonstrated by photocross linking with UV-sensitive amino acid precursors a physical interaction between cotransfected and affinity tagged mPR and both “insulin induced gene 1” protein (INSIG-1) and “sterol regulatory element binding protein (SREBP) cleavage-activating protein” (SCAP) in COS7 cells (Suchanek M, Radzikowska A, Thiele C, 2005, Photo-leucine and photo-methionine allow identification of protein-protein interactions in living cells. Nat Methods 2: 261-267).

At least one reagent is used for influencing, in particular, increasing or inhibiting the abundance and/or activity of isoforms of proteins for diagnosis and/or therapy of diseases associated with aberrant biological phenotypes, wherein the proteins are involved, in particular, differentially involved in protein interaction or multi-protein complexes with either phosphorylated or non-phosphorylated membrane associated progesterone receptor component 1 (mPR). With respect to further details, particularly with respect to phosphorylated or non-phosphorylated membrane associated progesterone receptor component 1 (mPR), it is referred to the above description.

Furthermore, at least one reagent is used for influencing, in particular, increasing or inhibiting the abundance and/or activity of proteins, in particular, of isoforms thereof, for the manufacture of a medicament and/or pharmaceutical composition for diagnosis and/or therapy of diseases associated with aberrant biological phenotypes, wherein the proteins are involved, in particular, differentially involved, in protein interaction or multi-protein complexes with either phosphorylated or non-phosphorylated membrane associated progesterone receptor component 1 (mPR). With respect to further details, particularly with respect to phosphorylated or non-phosphorylated membrane associated progesterone receptor component 1 (mPR), it is referred to the above description.

An assay kit is provided for diagnosis and/or therapy of diseases associated with aberrant biological phenotypes, comprising at least one isoform of membrane associated progesterone receptor component 1 (mPR).

An assay kit is provided for diagnosis and/or therapy of diseases associated with aberrant biological phenotypes, comprising at least one isoform of membrane associated progesterone receptor component 2 (PGRMC2).

The assay kit comprises means of detection and discriminating at least one isoform of membrane associated progesterone receptor component 1 (mPR) and/or PGRMC2 in at least one sample.

In the assay kit, the mPR may be phosphorylated mPR. The PGRMC2 may be phosphorylated PGRMC2.

The assay kit may comprise at least two isoforms of mPR in different phosphorylated status. The assay kit may also comprise at least two isoforms of PGRMC2 in different phosphorylated status. The assay kit may further comprise plasmids encoding mPR and/or mutants of mPR and/or mPR, which is expressed in cells, in particular, exogenously expressed. For a detailed explanation of a possible embodiment of such an assay kit it is referred to the further description.

A further aspect encompasses an assay comprising addition of reagents to an assay kit, comprising at least one isoform of membrane associated progesterone receptor component 1 (mPR) and analyzing the influence of the reagents on the phosphorylation status, i.e., the degree of phosphorylation, of mPR for diagnosis and/or therapy of diseases associated with aberrant biological phenotypes.

In addition, we provide pharmaceutical reagents developed by screening biological activity or effectiveness, in particular, to a pharmaceutical reagent screened by the inventive assay.

We discovered the association between the state of mPR phosphorylation and disease phenotype. Therefore, we use an assay comprising the determination of the phosphorylation status (degree of phosphorylation) of at least one isoform of membrane associated progesterone receptor component 1 (mPR) in mammalian samples, in particular, in human samples for diagnosis and/or therapy of diseases related to aberrant biological phenotypes.

According to a particular preferred example, the assay comprises screening for reagents that are suited for diagnosis and/or therapy of diseases that are associated with aberrant biological phenotypes. Regarding the reagents and diseases it is referred to the above description.

According to a further example, the human samples, preferably microdissected human samples, are derived from a small tissue fraction, particularly from a tumor tissue fraction, advantageously from a breast cancer tissue fraction. The human samples are preferably harvested by biopsy and/or surgical extraction.

Additionally, it is preferred to use the assay for determination of proteins that are involved, in particular, differentially involved in protein interaction or in multi-protein complexes, in particular, higher order multi-protein complexes, with either phosphorylated mPR or non-phosphorylated mPR. According to a further example, the assay is used for determination of proteins that are colocalized with phosphorylated, preferably hyperphosphorylated, mPR.

In a particular example, the membrane associated progesterone receptor component 1 (mPR) is of mammalian origin, in particular, of human origin, preferably human mPR (PGRMC1/Hpr6.6).

We designed a paired direct comparison strategy by pooling samples derived from tissue sections from large homogenous breast tumors on the basis being either ER⁺ or ER− negative. Eight ER⁺ tumors and eight ER⁻ tumors were used. They were randomly assigned to sub-pools (Table 1), each sub-pool containing normalized equal amounts of protein from two tumors. For differential analysis, sub-pool ER⁺¹ (containing T378 and T392) was differentially compared to sub-pool ER⁻¹ (containing T433 and T443), ER⁺² was compared to ER⁻², ER⁺³ was compared to ER⁻³, and ER⁺⁴ was compared to ER⁻⁴ (an example of one inverse replicate differential analysis is presented in FIG. 1). Spots were matched across gels, and their intensities were analyzed relative to ER status. Synthetic average gel images were constructed by computer (an example of which is given for the pH 5-6 experimental window in FIG. 2). The statistically most significant differential protein spots are preferably identified by mass spectrometry, in particular by MALDI-TOF (FIG. 3). In total, proteins from 325 spots were identified by MALDI-TOF PMF with MASCOT scores greater than 70, of which 72 spots represented 16 proteins that were identified in more than one protein spot (Table 2).

In a further step, we performed identification and characterization of proteins revealed by the above described study (Table 2), but which had not previously been directly linked to diseases associated with aberrant biological phenotypes, in particular subgroups thereof, especially cancer, preferably breast cancer.

According to a particular example, the revealed proteins were identified and characterized regarding their phosphorylation status. According to an especially preferred embodiment among the revealed proteins mPR was characterized by investigating its phosphorylation status in diseases associated with aberrant biological phenotypes, especially cancer, preferably breast cancer. The phosphorylation status (degree of phosphorylation) of mPR, in particular at least one isoform thereof, was investigated in subgroups of breast cancer, preferably in subgroups differing in the ER status (ER⁺ versus ER⁻).

In a particular example of use of the assay, the human samples are subjected to a radioactive labelling, in particular, to an inverse radioactive labelling, preferably with iodine isotopes. Preferably, an inverse radioactive labelling is performed using ¹²⁵I and ¹³¹I isotopes.

In a further example the assay is based on gel electrophoresis techniques, in particular SDS-PAGE (Sodium Dodecylsulfate Polyacrylamide Gel Elektrophoresis), especially two dimensional PAGE (2D-PAGE), preferably two dimensional SDS-PAGE (2D-SDS-PAGE).

According to a particular example, the assay is based on 2D-PAGE, in particular, using immobilized pH gradients (IPGs) with a pH range preferably over pH 4-9.

According to a further example of the assay, the gel electrophoresis techniques, in particular, the above mentioned techniques may be combined with other protein separation methods, particularly methods known to those skilled in the art, in particular, chromatography and/or size exclusion.

If appropriate, the above mentioned methods may be combined with detection methods, particularly known to those skilled in the art, in particular, antibody detection and/or mass spectrometry.

Since the difference between the mPR isoforms under consideration is due to the presence of phosphate groups, all methods enabling the detection of subtle or extreme differences in the stoichiometry of phosphate or oxygen atoms in proteins are within the scope of this disclosure. In this regard, in particular, preferred methods may be elemental analysis, measurement of the state of ionization or differential electrical conductivity. According to a further example, methods enabling the measurement of differences in the stable isotope content of proteins, in particular, of chemically modified proteins, or degradation products thereof are part of this disclosure.

According to a particular aspect of the assay, affinity reagents are applied, in particular, antibodies. For instance, the affinity reagents, in particular, antibodies may be used in an immunoassay, particularly in an ELISA (Enzyme Linked Immunosorbent Assay).

In a further aspect, the assay comprises application of mass spectrometry, in particular, MALDI (Matrix Assisted Laser Desorption/Ionization) and/or SELDI (Surface enhanced Laser Desorption/Ionization).

Resonance techniques, in particular, plasma surface resonance, may be used.

In some cases, it may be advantageous to achieve a separation of proteins, preferably of mPR, in particular, by means of one of the above outlined examples before cleaving the proteins. Such a cleavage step can be performed by applying enzymes, chemicals or other suitable reagents which are known to those skilled in the art. As an alternative, it may be desirable to perform a cleavage step before separation of the peptides, in particular, of mPR, obtained by the cleavage step followed preferably by measurements of mPR concerning its abundance and/or degree of phosphorylation.

According to a further example, the labelled and, in particular, separated protein spots are visualized by imaging techniques, for instance by the Proteo Tope® imaging technique.

We identified membrane associated progesterone receptor component 1 (mPR) from three spots (Table 2) that formed an approximately equidistant chain in the pH 4-5 IPG, two of which (FIG. 3: spots 52, and 62) were significantly more abundant in cancers with a negative ER status (ER⁻). Furthermore, the more acidic spots exhibited slightly retarded migration in SDS-PAGE (Sodium Dodecylsulfate Polyacrylamide Gel Elektrophoresis), consistent with possible phosphorylation differences between the spots. The putatively hypophosphorylated forms were more abundant in tumors lacking the estrogen receptor. To test the hypothesis that these quantitative differences were due to altered isoelectric points of the protein caused by differential phosphorylation in the spots, and that phosphorylation therefore may differentially affect the intracellular localization of mPR between the test tissues, we devised a phosphatase treatment regime using shrimp alkaline phosphatase (SAP). Whole cell native protein extracts from several patients were pooled and incubated with either phosphatase buffer containing SAP (+SAP), or mock incubated under identical conditions with the addition of phosphatase inhibitors but without SAP (−SAP). A raw extract that was not incubated at all prior to protein denaturation (raw) was also included in the analysis as a reference control.

For instance, we analyzed the samples pairwise against each other by ProteoTope® imaging after inverse radioactive labelling with ¹²⁵I and ¹³¹I, and separation by daisy chain 2D-PAGE. The portions of the part of the inverse replicate gels containing the mPR spots are shown in FIG. 5. Panel A, being ¹²⁵I-labelled +SAP sample (blue) and ¹³¹I-labelled mock −SAP sample (orange) shows a discernable preponderance of ¹³¹I for the most acidic spot (spot 38). By contrast, the most basic of the spots (spot 52) exhibited a slight preponderance of ¹²⁵I. Importantly, these small differences in relative signal intensity were reproducibly detected in the inverse replicate labelled experiment of Panel B, where spot 38 also shows a discernable preponderance of ¹²⁵I and spot 52 of ¹³¹I. Panels B and C compared the phosphatase treated samples against the untreated raw extract. The same trend was observed. However, the magnitude of the differences was slightly higher, the difference being possibly due to experimental error. By contrast, when the mock treatment was compared to the raw extract in panels E and F, the ratios between both samples approximated 50%. Thus, the difference in intensity of this spot was not due to the incubation, but rather due to the presence of phosphatase in the incubation. The averaged quantified results from both inverse replicate dual image gels for each sample comparison are presented graphically in Panel G. This result strongly demonstrates that the most acidic spots can be dephosphorylated, whereupon they migrate to one of the more basic spots. Taken together with the results of FIG. 3 for these three protein spots, this provided evidence that mPR is probably more highly phosphorylated in ER⁺ than ER⁻ tumors.

Therefore, we provided evidence that the population of mPR molecules is more highly phosphorylated in ER⁺ tumors than ER⁻ tumors. Consequently, this is the first de-novo demonstration of a phosphorylation difference from primary tumors by discovery proteomics without the use of cell culture. Interestingly, this phosphorylation pattern corresponds to the presence of punctuated localized concentration of mPR in extra-nuclear regions of ER⁻ cells (FIG. 4).

We further examined potential protein motifs in Table 3 for mPR (SwissProt entry O00264). For a protein of just 194 amino acids (21.5 kDa), in addition to the cytochrome b₅ domain, a plurality of predicted short motifs concerned with protein interactions and signal transduction molecules is present, including two SH2 domains, an SH3 domain, a tyrosine kinase site, two CK2 sites, and consensus binding sites for ERK1 and PDK1. FIG. 9 shows the position of the predicted ScanSite MotifScan motifs (Obenauer J C, Cantley L C, Yaffe M B. 2003. Scansite 2.0: Proteome-wide prediction of cell signaling interactions using short sequence motifs. Nucleic Acids Res. 31: 3635-41) in the PGRMC1 sequence. Furthermore, these are all on the surface of the folded protein. Although not all of these sites may be biologically relevant, this nevertheless strongly suggests that mPR may be able to function as an adaptor molecule in signal transduction processes.

We noted that both of the acidophilic kinase (for convenience referred to as “CK2 sites,” without meaning to imply that CK2 is necessarily the kinase involved) have been detected as phosphoserine peptides in mPR from HeLa cell nuclear extracts (Beausoleil S A, Jedrychowski M, Schwartz D, Elias J E, Villen J, Li J, Cohn M A, Cantley L C, Gygi S P. 2004. Large-scale characterization of HeLa cell nuclear phosphoproteins. Proc Natl Acad Sci USA. 101: 12130-5; Table 3). Additionally, the presence of higher molecular weight species of mPR in MCF7 breast cancer cell line were observed by Selmin et al. (Selmin O, Thorne P A, Blachere F M, Johnson P D, Romagnolo D F, 2005, Transcriptional activation of the membrane-bound progesterone receptor (mPR) by dioxin, in endocrine-responsive tissues. Mol Reprod Dev 70: 166-174). Confirming us, those authors speculated that the species may have represented differentially phosphorylated states of mPR, which would cause changes in the apparent molecular weight. Particularly, this explanation would be in line with the fact that RT-PCR experiments detect one single transcript, and previous Northern assays which showed a single mPR RNA size, corresponding to a protein of ≈25 kDa.

Considerable information is available concerning the structure and homologous conservation of the cytochrome b₅ domain proteins. Indeed the predicted sites from Table 3 exhibit high conservation, sometimes from yeast to mammals. We aligned the conserved structural beta strands and helical elements to the sequence of mPR, including the predicted protein motifs from Table 3 (SEQ ID NO:1-8). Remarkably, the two CK2 sites flanked the conserved cytochrome b₅ domain (SEQ ID NO: 10), and each of these overlapped with a predicted SH2 and SH3 MotifScan target sequence respectively (FIG. 6, SEQ ID NO:9). This immediately suggests the possibility that CK2 phosphorylation could negatively regulate protein interactions mediated by these target sequences, and/or that phosphorylation by CK2 could allosterically affect the conformation of the ligand binding domain. To assess this, we examined two available protein structures: the bovine cyt-B5 structure (Durley and Mathews, 1996) and the cytochrome b₅-domain Arabidopsis protein “Putative Steroid-Binding Protein” (Genbank Accession GI:40889041) for which an NMR structure has been submitted by Suzuki et al. in 2002 to the Research Collaboratory for Structural Bioinformatics (RCSB: http://www.rcsb.org/pdb/) Protein Data Base (PDB) with PDB Accession 1J03. Alignment of the cytochrome b₅ domain of the Arabidopsis protein with mPR confirm that the major structural elements are conserved, importantly, including the approximate number and partial identity of amino acids in the putative SH2 domain between Helices 3 and 4 (FIG. 7). Many of the non-identical amino acids within the beta strands or alpha helices furthermore represent conservative substitutions. This demonstrates that these proteins are structurally homologous over this cytochrome b5 domain, and that the NMR structure of 1J03 can validly be superposed with the amino acid backbone of mPR and related proteins to help understand their structure/function relationships.

The structures of these two proteins are presented graphically in FIG. 8, showing the structural motif labelling nomenclature from FIG. 6 according to the mPR amino acids in SwissProt Accession 000264. A model for mPR is shown in FIG. 9, based upon homology to Arabidopsis 1J03 (FIG. 7). Using the aligned domain topology of these two protein structures, we noted that the N-terminal and C-terminal regions of both cytochrome b₅ domains were on the opposite side of the protein than the ligand binding domain. Therefore, the predicted mPR N-terminal SH3 target sequence and CK2 site centered on residues P62 and S62, respectively (Table 3), are most probably on the surface, in particular, adjacent to one another, of the related mPR protein directed away from the ligand binding pocket. Similarly, the predicted SH2 target sequence and CK2 consensus site centered on Y179 and S180, respectively, are also probably located away from the ligand binding site. This suggests that ligand binding of mPR could occur simultaneously with protein interactions through these sites. We also observed that the predicted Shc consensus-like SH2 target sequence was inserted as a loop between conserved cytochrome b₅ domain helices 3 and 4 (H3-H4 SH2 in FIG. 9) in the MAPR family that is absent in the ancestral cytochrome b5 domain fold. In the crystal structure of bovine cyt-b5, these two helices are joined by a short linker at the rim of the ligand binding domain. In the plant protein the amino acids between helices 3 and 4, which exhibit marked amino acid homology to the predicted SH2 motif of mPR, fold to the side away from the ligand pocket, leaving the ligand binding domain accessible. Furthermore, the region of putative Y138 phosphate acceptor amino acid of mPR exhibits in the plant protein 1J03 several amino acids in the putative SH2 target loop having absolute conservation, and several other amino acids having undergone conservative substitutions with strong homology across this region.

Interestingly, structural alignment of mPR with particularly the plant protein also revealed that P108 at the center of the putative ERK1 binding site (Table 3) was exposed at the surface of the protein immediately N-terminal to helix 2° (FIG. 6), while T160 at the centre of the putative PDK1 kinase binding site was also exposed on the surface of helix 4. In fact, the P108 putative ERK1 binding site is situated on the lip of the binding pocket adjacent to the position where a cyt-B5 histidine interacts with the heme ligand, and this proline is conserved in related proteins from yeast to mammals. Therefore the putative RK1 binding could conceivably be inhibited by recruitment of the ligand to the binding pocket, or vice versa. Similarly, a predicted phosphorylation of Y112 by Abl or Lck kinase could also potentially influence ligand binding. We noted that all of the YXX(Φ)/ITAM potential motifs described are actually accessible at the protein surface on sharp turns in the modelled mPR structure, which is the structural prerequisite for involvement in vesicle trafficking. The functional relevance of these putative structural motifs certainly merits further scrutiny, and it is reasonable to accept that this distribution could not have arisen by chance, and that therefore mPR is involved in membrane trafficking. Furthermore, the kinase PDK1 (3-phosphoinositide-dependent protein kinase-1) phosphorylates and activates kinases that are regulated by PI 3-kinase, which in turn regulate cellular metabolism, growth, proliferation and survival, all of which may be related to the biology of ER− tumors. The functional relevance of these putative kinase binding sites, and the putative involvement of these kinases in the breast cancer-associated biology of mPR, merit further scrutiny.

Additionally, we conducted cell culture experiments with stable and transient transfected cells, respectively, using plasmids encoding wild type and mutant mPR proteins, respectively, and observed phenotypes.

For this purpose, the MCF7 breast cancer cell line and plasmid pcDNA3.1 expressing a C-terminally tagged PGRMC1 protein were used. The C-terminal tag consisted of three repeats of amino acids from the heamaglutinin protein, generating the protein (PGRMC1-3HA).

We observed differences in phosphorylation status between estrogen receptor positive tumors and estrogen receptor negative tumors of the breast. Since the prognosis for estrogen receptor-negative tumors is quite poor, with those tumors exhibiting resistance to treatment and causing high mortality levels, we reasoned that mPR phosphorylation status contributed to the resistance to treatment.

Bioinformatics analysis revealed the presence of potential phosphate acceptor sites in association with the recognition motif sites for SH2 and SH3 domain-containing interaction partners, as described. Accordingly, the potential phosphate acceptor amino acids shown in FIG. 10 on the protein surface were mutated to chemically related amino acids that should exert only minimal influence on protein folding. The mPR open reading frame (ORF) was present in the eukaryotic plasmid expression vector pcDNA3_MPR_{—}3HA. The nucleotide sequence of pcDNA3_MPR_{—}3HA is shown as SEQ ID 11. The amino acid mutations that were introduced into the ORF of mPR by site-directed mutagenesis of plasmid pcDNA3_MPR_{—}3HA are shown in FIG. 10, and the amino acid sequence of the mutated proteins, are shown in SEQ ID 12 to SEQ ID 20. The codon changes that were introduced to accomplish these mutations are presented in Table 4. The same effects are obtainable using mPR that lacks a hemaglutinin (HA) amino acid tag. Although not all possible mutants are shown in this example in principle all mPR variants are in the scope herein, including all mammalian homologues of mPR, and notable the related family member PGRMC2.

### Stable Cell Transfections

Colonies were grown from all transfected cell lines. Surprisingly, the number of colonies was drastically reduced to effectively background levels with mutant S56A/S180A and mutant Y179F/180A as can be seen from cell counting results in Table 5 and FIG. 12, and as shown in the colony forming assay of FIG. 11. Surprisingly, there was a reduction in the number of viable cells transfected with mutant S56A/S180A and mutant Y179F/S180A. These results demonstrate that mutating serine 56 and serine 180, or Y179 and S180 has a profound effect on either proliferation or apoptosis of effectively all MCF-7 cells. This anti-proliferative effect of both of these mutants was not observed using transiently transfected cells used for immunohistochemistry in cytospins because these were assayed shortly after transfection.

The antiproliferative effect of the S56A/S180A mutant PGRMC1 molecule was dependent upon the presence of Cysteine 128, since the triple mutant S56A/C128S/S180A was able to proliferate. These data indicate that the disulfide-linked dimeric form of mPR that has been reported has a dominant inhibitory effect on cell proliferation, and this effect requires a hypophosphorylated form of mPR and involves the cysteine residue. Therefore, treatments that would promote the formation of the hypophosphorylated dimeric form in tumors would be highly desirable. The results indicate that serine 180 imposes an antiproliferative effect when it is unphosphorylated, and when either tyrosine 179 or serine 56 is also unphosphorylated. Therefore the interaction of an SH3-domain-containing protein that interacts with the SH3-domain target sequence that contains serine 56 is necessary to manifest the antiproliferative phenotype associated with non-phosphorylated serine 180. The interaction of this unknown protein is inhibited by phosphorylation of serine 56, thereby preventing the antiproliferative phenotype and enhancing proliferative ability. Conversely, when an SH2-domain-containing protein can bind to the phosphorylated tyrosine 179 adjacent to non-phosphorylated serine 180 then the antiproliferative effect is antagonized, and cells can proliferate. Phosphorylated serine 180 also abrogates the antiproliferative effect of mPR, which raises the possibility that perhaps a single protein which interacts with the mPR C-terminal SH2 target sequence can recognize a phosphate on either tyrosine 179 or the adjacent serine 180.

Therefore, the antiproliferative effect requires a protein that interacts with the non-phosphorylated SH3 target sequence of mPR when it is in the dimeric form. This SH3 target sequence is similar to the SH3-binding region of interleukin and cytokine receptors (Selmin et al., 1996. Carcinogenesis. 17: 2609-15) that recruit SH3-containing JAK kinases (Tanner et al., 1995. J Biol. Chem. 270: 6523-30). These kinases are activated by binding to dimerized proteins such as cytokine receptors because their effective concentrations relative to each other are increased, and the two bound kinases then reciprocally phosphorylate one another because of the resulting increased enzymatic activity (which is proportional to concentration). In this respect it is notable that the SH3 consensus target sequence centered upon proline 62 corresponds well to the sequence requirements for binding to ABL kinase (Score 0.5081, which is in the best 0.907% of sites from http://scansite.mit.edu/motifscanner/motifscanl.phtml, SwissProt sequence O00264), and JAK kinases are activated by ABL kinase (Danial & Rothman. 2000. Oncogene. 19: 2523-31).

### Transient Cell Transfections

The plasmids containing mPR variants, wild-type and mutants as shown in FIG. 10 were separately transfected into MCF7 human breast cancer cells under identical conditions. As can be seen from FIG. 13, wild-type mPR exhibited a perinuclear cytoplasmic distribution, due to the fact that cytoplasm was relatively small in volume. The transient expression of mPR with mutations of either serine 56, serine 180, or both, to alanine (mPR mutants S56A, S180A, or S56A/S180A) resulted in increased frequency of enlarged cells with well developed cytoplasmic organization and a cytoplasmic and cytoplasmic-membrane distribution of mPR. The gross cellular localization of the double mutant S56A/S180A was not markedly affected by introduction of the third mutation of cysteine 128 to serine (S56A/C128S/S180A). Strikingly, none of these cells exhibited the phenotype of MCF7 cells transfected with the parental vector expressing HA-tagged wild-type mPR. Therefore the presence of Serine 56 and Serine 180 is necessary to maintain a higher frequency of the condensed phenotype of MCF7 cells transfected with mPR. Furthermore, the mPR phosphorylation-site mutants affect cellular morphology, which is a striking demonstration that the phosphorylation of mPR is able to influence cancer cells.

We observed a common phenotype of low cytoplasmic mass and perinuclear mPR localisation when wild-type mPR was overexpressed in MCF7 cells. However, it was demonstrated that different phenotypes are in fact dictated by the phosphorylation status of mPR. In the absence of serine 56 or serine 180 the low cytoplasmic mass phenotype with perinuclear mPR was not observed. The different mutants revealed the involvement of mPR in an anti-proliferative pathway that is regulated by phosphorylation. Since this reflects the biological role of mPR, we are the first to demonstrate the role of mPR in cancer processes, the first to demonstrate that mPR is phosphorylated, the first to demonstrate that mPR phosphorylation states differ between clinically relevant classes of tumors, and the first to demonstrate that the ability to be phosphorylated at different positions correlates with the ability of cells to proliferate. Therefore, the differentially phosphorylated forms of mPR observed in breast cancers from patients reflect the biology that is described according to this disclosure. This demonstrates the validated involvement of mPR in human cancers, and the mechanistic basis of the involvement.

Immuneprecipitation of DCC with mPR

Runko and Kaprielian (Runko E, Kaprielian Z, 2004, Caenorhabditis elegans VEM-1, a novel membrane protein, regulates the guidance of ventral nerve cord-associated axons. J Neurosci 24: 9015-9026) demonstrated that VEM-1, the nematode homologue of mPR, physically interacts with C. elegans UNC-40, the nematode homologue of the mammalian cell surface receptor “deleted in colorectal cancer” (DCC) (human SwissProt P43146, NM_{—}005215). Therefore it is most reasonable to assume that mPR will be detectable in a complex with DCC. The hypothesis was tested that mammalian DCC/Unc-40 was present in a protein complex with mPR, which has previously been demonstrated only for the C. elegans homologues of these proteins. Aliquots of 2×10⁶ MCF7 cells were transfected as described below with each of the mPR expression vectors.

After 24 hours the mPR-3HA proteins were affinity purified with an anti-HA anti-body described above. The AF5 anti DCC human monoclonal antibody (Merck Biosciences OP45 Anti-DCC Mouse mAb AF5) was used to detect the presence of DCC by Western Blot. Normalized amounts of the precleared incubation reaction, the final supernatant after removal of the immuno-precipitate, and of the immuno-precipitated pellet were loaded to SDS-PAGE gels, blotted to membranes, and Western Blotted with the AF5 anti-DCC antibody. Results are shown in FIG. 14. A high molecular weight DCC band was observed in immune precipitation pellets of wild-type mPR, but from none of the mutants. The size of the band was slightly higher than the predicted 158 kDa for DCC, possibly due to post-translational modification of the protein. An approximately 100 kDa band also reacted with the anti-DCC monoclonal antibody in Western Blot, which may represent a proteolytic fragment of DCC. This represents the first demonstration of a physical interaction between mPR and DCC in mammalian cells, and simultaneously demonstrates that the interaction requires both serine 56 and serine 180 of mPR. We are not naively claiming a phosphorylated form of mPR interacts with DCC, which is not shown by our data. It is possible a non- or hypo-phosphorylated form of mPR protein actually interacts with DCC; with phosphorylations rather determining the subcellular or extracellular localization that is permissible for DCC-interaction.

In view of the above mentioned observations, a model for the cellular function of mPR in breast cancer was considered by us. As stated above, the preponderance of predicted protein motifs associated with signal transduction in the 21.5 kDa protein shows that the protein indeed functions as a signalling adaptor molecule that directs the sub-cellular location of interacting proteins and membranes in response to signals, and that the activity may be ligand-regulated, in particular be steroid- or cholesteroid-responsive, or responsive to other ligands, such as heme. An interplay between ligands is a possible mechanism of regulation which offers potential avenues of pharmaceutical intervention. Strikingly, the spatial juxtaposition of the SH2 and SH3 target motifs in FIG. 8 and in FIG. 9 would induce intimate local concentration of potential interacting proteins relative to each other, such as possibly kinases and their substrates, or proteins bound to different subcellular membranes, potentially greatly facilitating enzymatic activities and biological actions. This provides an explanation for the mode of action of mPR. The protein is schematically represented in FIG. 15A as a ligand binding signalling adaptor protein. In the ER⁺ state, one or more of the N-terminal SH3 target sequence and C-terminal SH2 target sequence are phosphorylated by CK2 or related kinases, in particular acidophilic kinases, and do not interact with other proteins. Since the C-terminal CK2 site is more highly evolutionarily conserved, and overlaps exactly with the corresponding SH2 target sequence, it is the more likely of these motifs to be functionally regulated by CK2, although both sites have been observed to be phosphorylated. However in the non-CK2-phosphorylated state, in particular in the ER⁻ state, the CK2 site/s is/are dephosphorylated, leading to relocation of mPR to specific subcellular compartments, as observed in FIG. 4. The SH2 target sequence between helices 3 and 4 of the cytochrome b5 domain may interact with other proteins in both cell types. However interactions through this domain may be regulated by ligand binding, tyrosine phosphorylation of the Abl/Lck-like site at Y112, and/or binding of either ERK1 and/or PDK1 to their respective sites at the ligand pocket. FIG. 15B depicts one possible model for mPR in ER⁺ cancers. The protein exists predominantly in the state phosphorylated by the constitutive kinase CK2, preventing the predicted protein interactions through the SH3 and SH2 domains that flank the cytochrome b₅ domain. In FIG. 15C the CK2 sites are dephosphorylated in ER⁻ cancers, leading to interaction with other proteins, sub-cellular relocalization, and activation of signal transduction or more generally an alternative cellular behaviour. The present results reveal significant differences in mPR between ER⁺ and ER⁻ tumors for the first time, and further reveal that this protein is potentially a potent target for in particular anticancer therapy of ER⁻ tumors.

Further advantages, features and possible uses are described below by means of the examples with reference to the above described tables and figures. In this connection, the various features may in each case be implemented on their own or in combination with one another.

### Materials and Methods

Patients and tissue samples. Primary breast cancer specimens were obtained with informed consent from patients, who were treated at the Department of Gynecology and Obstetrics, University Hospital Tübingen (Ethikkommission Med. Fakultät AZ.266/98). Samples were characterized and collected by an experienced pathologist. After removal of breast tumor from the patient, the tissue samples were embedded in O.C.T. compound (Leica), then snap frozen in liquid nitrogen within 15 minutes of tumor removal, and stored at −196° C. in a tumor tissue bank. Sample collection was approved by an ethics committee and by the patient. Tumor data were stored in an Oracle-based database according to practices approved by the Institute of Electrical and Electronics Standards Association (IEEE-SA). Clinical information was obtained from medical records and each tumor was diagnosed by a pathologist, according to histopathological subtype and grade. The tissue quality of each tumor was verified by measuring RNA integrity from one or more slices with an Agilent 2001 Bioanalyser. Tumors lacking sharply distinct 18S and 28S ribosomal RNA bands were excluded from the study. ER, PR and HER-2/neu status for each tumor were routinely determined by immunohistochemistry.

### Preparation of Cryosections

Tumor samples were selected using the database, removed from the tissue bank on frozen CO₂ and transferred to a cryotome (Leica) at a temperature of −23° C. Cryogenic sections (10 μm) were subsequently sliced, placed on SuperFrost+-slides (Multimed) and stored at −80° C. until further use. For immunopathologic characterization by an experienced pathologist one section was stained with hematoxilin/eosin.

### Proteomics Analysis

ProteoTope® analysis was performed essentially as described (Neubauer H, Clare S E, Kurek R, Fehm T, Wallwiener D, Sotlar K, Nordheim A, Wozny W, Schwall G P, Poznanovic S, Sastri C, Hunzinger C, Stegmann W, Schrattenholz A, Cahill M A. 2006. Breast cancer proteomics by laser capture microdissection, sample pooling, 54-cm IPG IEF, and differential iodine radioisotope detection. Electrophoresis. 27: 1840-52.). Frozen tumor sections of 10 μm were lysed directly into SDS buffer, separately iodinated in inverse replicated with each of I-125 and I-131, and separated by 54 cm daisy chain IEF-IPG after sample pooling as described in Table 1. Aliquots of each sample were each iodinated by either ¹²⁵I, or ¹³¹I, respectively, using approximately 6 MBq of each isotope per 3.6 μg pooled sample aliquot under identical chemical conditions in a reaction volume of 25 μL by the iodogen method as described. Radioactive iodine was purchased from Amersham Biosciences (Freiburg). 2D-PAGE was performed using 18 cm commercial immobilized pH gradients (IPGs) in serial 54 cm IPG-IEF over pH 4-9 (pH 4-5; pH 5-6; pH 6-9) that were run in the SDS-PAGE dimension as 3×18 cm IPGs in a Hoefer ISO-DALT.

### Shrimp Alkaline Phosphatase (SAP) Analysis

Cryogenic slices from 6 patients (30 slices T433, 40 slices T443, 40 slices T469, 40 slices T470, 35 slices T623, 30 slices T640) were each extracted with 200 μL aliquots of SAP-dephosphorylation buffer (50 mM Tris pH 8.5, 5 mM MgCl₂, 0.25% CHAPS, supplemented with 1×EDTA-free Complete protease inhibitor cocktail from Roche); This precooled buffer was added directly on ice to the frozen slices in eppendorf tubes and the tissue was mechanically homogenized using a plastic pellet pestle. Tubes were vortexed and incubated for 30 min at 4° C., followed by centrifugation for 15 min at 14 000×G at 4° C. Supernatants were collected together and the protein concentration was assayed by the BCA method as described. The yield was approximately 4 mg of protein. 30 Units of SAP in 30 μL were added into 800 μg of protein in 400 μL of in SAP-Dephosphorylation buffer, followed by mixing and incubation for 16 h at 37° C. In parallel a mock incubation control was performed on 800 μg of protein in the same buffer without the addition of SAP, and containing the following phosphatase inhibitors: activated vanadate, sodium fluoride, and sodium glycerophosphate at final concentrations of 1 mM, 5 mM, and 5 mM respectively. The incubation was performed in parallel at 37° C. for 16 h. Following incubation the proteins were frozen at −80° C. A non-incubated raw lysate control containing 800 μg of protein in 400 μl of SAP buffer was frozen at −80° C. without additions or incubation. Frozen protein mixtures were thawed, precipitated, and resuspended at 1 μg/μl in boiling 0.1M Tris, 2% SDS, pH8.5. 60 μg of protein were then used for iodination with each of 1-125 or 1-131 as described. Differential inverse replicate ProteoTope analysis was as described above for 54 cm daisy chain IPG-IEF after rehydration loading overnight to the pH 5-6 IPG.

### Protein Identification by Mass Spectrometry

Protein identification is based on different mass spectrometric methods: an automated procedure that allows a very quick and reliable identification of higher abundant proteins (peptide mass fingerprinting with MALDI-TOF-MS) but also allows the identification of very low abundant proteins with more time consuming procedures (LC-ESI-IonTrap-MS/MS, or MALDI TOF-TOF). Briefly, gel plugs of selected protein spots are excised and the proteins contained in the gel plugs are digested using trypsin. The resulting solution is analyzed first with a high throughput peptide mass fingerprint procedure based on MALDI-TOF-MS. For those spots where only ambiguous identification was achieved, a fragment ion analysis based on MALDI TOF-TOF or LC-ESI-IonTrap-MS/MS was added. A detailed description of typical MALDI-TOF-MS procedures has been published (Vogt J A, Schroer K, Holzer K, Hunzinger C, Klemm M, Biefang-Arndt K, Schillo S, Cahill M A, Schrattenholz A, Matthies H, Stegmann W. 2003. Protein abundance quantification in embryonic stem cells using incomplete metabolic labelling with 15N amino acids, matrix-assisted laser desorption/ionisation time-of-flight mass spectrometry, and analysis of relative isotopologue abundances of peptides. Rapid Communications in Mass Spectrometry, 2003; 17: 1273-1282).

### Database Searching

For the identification of the proteins the peptide masses extracted from the mass spectra were searched against the NCBI non-redundant protein database (www.ncbi.nlm.nih.gov) using MASCOT software version 1.9 (Matrix Science, London, detailed description can be found at http://www.matnixscience.com/).

### Site-Directed Mutagenesis

Mutations of specific amino acids in pcDNA3.1-PGRMC₁₋₃HA were generated according to standard methods by commercial service providers.

### Cell Culture Experiments

### Stable Transfections

MCF-7 cells stably transfected with mPR and mutants, respectively, were established. 5 μg of expression plasmid pcDNA3.1 containing hemaglutinin (HA)-tagged mPR WT or HA-tagged mutants S56A, S180A, S56A/S180A, S56A/C128S/S180A, Y138F, Y179F or Y179F/S180A were transfected into MCF-7 breast cancer cells. For transfections a transfection device and transfection kits from AMAXA Biosystems were used (Gaithersburg, Md., USA) according to the manufacturer's recommendation. 2×10⁶ cells were transfected with circular plasmids and plated with RPMI-Medium for 24 h. Then Medium was changed to RPMI complete medium containing 60 μg/ml hygromycin B and cells were cultured for 2 weeks for selection of stable integration events. After two weeks single colonies had formed and limiting dilution assays were performed to select for colonies grown from a single cell. To that aim colonies were trypsinized, counted and diluted in two-fold dilutions.

### Transient Transfections

MCF-7 breast cancer cells were transfected with 5 μg of pcDNA3.1 containing HA-tagged mPR, tagged mutant A, B, C, or tagged mutant D. The AMAXA transfection system (see above) was used according to the manufacturer's recommendation. After transfection of 2×10⁶ cells the cells were split into 3 wells from a 12-well plate (3.2 cm²). Cells were grown for 24 h. Then medium was changed to RPMI without phenol-red, 5% Hyclone stripped FCS and 1% Penicillin/streptomycin to starve cells for hormones contained by normal FCS. Cells were incubated for 24 h. Afterwards cells were washed with PBS, trypsinized and counted. Cytospins were prepared using 5×10⁵ cells per slide.

### Immune Fluorescence

Cytospins were air dried overnight and then fixed with 0.05% formalin, washed with PBS, treated on ice with PBS/0.1% Triton for 15 min and washed again with PBS. To avoid background labelling, cytospins were blocked with 10% normal serum according to the species of the secondary antibody (here: goat). After removal of the block primary anti-HA-specific antibody (rabbit, Santa Cruz) was applied in a 1:100 dilution in antibody diluent (Dako Norden A/S, Glostrup, Denmark) and incubated for 1 h in a humid chamber at room temperature. The cytospin was washed with phosphate buffer (phosphate buffered saline, PBS) once and then secondary goat anti-rabbit-Alexa Flour 594 antibody (Molecular Probes) was used to detect the primary antibody. Anti cytokeratin antibody was analogously visualized by fluorescein isothiocyanate (FITC). DNA staining was by DAPI (4′,6′-diamidino-2-phenylindole). After 30 min in the humid chamber the cytospin was washed twice with PBS. The cytospins are not allowed to dry. For staining the nucleus VECTASHIELD® Mounting Medium (Vector Laboratories Inc., Burlingame, Calif., USA) was used for embedding the cytospins under a coverslip. Fluorescence was detected using a Metafer4 fluorescence scanning microscope (MetaSystems GmbH, Altussheim, Germany) and the accompanying “Isis” software provided by MetaSystems. Results are shown in FIG. 13.

### Immunoprecipitation (IP)

MCF-7 breast cancer cells were transfected by Lipofection with 5 μg of pcDNA3.1 containing HA-tagged mPR-wt, tagged mutant S56A, S180A, S56A/S180A, or tagged mutant S56A/C128S/S80A. After transfection of 2×10⁶ cells they were cultured in RPMI-Medium with 5% FCS. For IP the cells were trypsinized, counted and cell pellets were snap frozen and stored at −80° C.

For IP the pellets were lysed in lysis buffer (M-Per Mammalian Protein Extraction Reagent+Halt Protease Inhibitor Cocktail Kit, PIERCE) and preclearance was performed with Protein A Sepharose CL-4B (Amersham) beads (preincubated with rabbit normal serum) for 1 h at 4° C. Beads were separated by centrifugation and stored at −80° C. To affinity purify the HA-tagged PGRMC1 variants these lysates were further incubated with Protein A Sepharose CL-4B preincubated for 1 h at room temperature with polyclonal rabbit anti-HA-antibody (Santa Cruz). Incubation was performed for 16 h at 4° C. in a rotating tube. Then, beads were separated by centrifugation and washed twice with ice cold lysis buffer.

For PAGE gel-loading buffer/mercatoethanol was added to beads and the final supernatant, heated to 95° C. for 5 min and loaded on a 10% polyacrylamide gel. After separation of the proteins, western blot was performed onto nitrocellulose membrane. Transfer was controlled by Ponceau-red staining visualizing protein bands. After destaining the membranes were blocked for 16 h at 4° C. with 5% milkpowder/0.05% Tween. The next day membranes were incubated with mouse monoclonal anti-DCC antibody (1:20) (Biozol/Abcam) for 2 h at RT followed by biotinylated anti-mouse IgG antibody (Vecta Stain). For detection, membranes were incubated with streptavidin/HRP complex (DAKO) for 2 h at RT followed by enhanced chemiluminescence (ECL) treatment and measurement of chemiluminescence using a Lumiimager (Roche). As a loading control, membranes were incubated with rabbit polyclonal Actin I-19 antibody (Santa Cruz).

1-17. (canceled)

18. A method of diagnosing diseases associated with aberrant biological phenotypes comprising:
determining or estimating the degree of phosphorylation of at least one isoform of membrane associated progesterone receptor component 1 (mPR) in a sample to be tested.

19. A method of diagnosing diseases associated with aberrant biological phenotypes comprising:
determining or estimating the degree of phosphorylation of at least one isoform of membrane associated-progesterone receptor component 2 (PGRMC2) in a sample to be tested.

20. The method according to claim 18, wherein the degree of phosphorylation is determined and/or estimated by analyzing proteins that are differentially involved in protein interaction or multi-protein complexes with either phosphorylated or non-phosphorylated membrane associated progesterone receptor component 1 (mPR).

21. The method according to claim 18, wherein the degree of phosphorylation status of the mPR is determined by antibody affinity reagents.

22. The method according to claim 18, wherein the mPR is derived from mammalian samples.

23. The method according to claim 21, wherein the sample is harvested by biopsy and/or surgical extraction.

24. An assay kit that diagnoses and/or treats diseases associated with aberrant biological phenotypes comprising at least one isoform of membrane associated progesterone receptor component 1 (mPR).

25. The assay kit according to claim 24, wherein the mPR is phosphorylated mPR.

26. The assay kit according to claim 24, comprising at least two isoforms of mPR in different phosphorylated status.

27. The assay kit according to claim 24, comprising plasmids encoding mPR and/or mutants of mPR and/or mPR, expressed in cells, and exogenously expressed.

28. The assay kit according to claim 24, comprising at least one isoform of membrane associated progesterone receptor component 1 (mPR) and analyzing the influence of added reagents on the degree of phosphorylation mPR for diagnosis and/or therapy of diseases associated with aberrant biological phenotypes.

29. The method according to claim 18, further comprising increasing the degree of phosphorylation with at least one reagent.

30. The method according to claim 18, further comprising inhibiting the degree of phosphorylation with at least one reagent.

31. A method of treating diseases associated with aberrant biological phenotypes comprising administering a therapeutically effective amount of at least one reagent that influences the degree of phosphorylation of at least one isoform of membrane associated progesterone receptor component 1 (mPR) to a mammal.

32. The method according to claim 18, wherein the at least one isoform is phosphorylated or non-phosphorylated.

33. The method according to claim 31, wherein the diseases comprise cancer, neurodegenerative diseases, infertility, inflammatory, immunological, respiratory, pulmonary diseases, and/or diseases associated with the rate of biological aging or with beneficial or detrimental alterations of the level of the process of autophagy.

34. The method according to claim 33, wherein the cancer is breast cancer or prostate cancer.

35. The method according to claim 19, wherein the diseases comprise cancer, neurodegenerative diseases, infertility, inflammatory, immunological, respiratory, pulmonary diseases, and/or diseases associated with the rate of biological aging or with beneficial or detrimental alterations of the level of the process of autophagy.

36. The method according to claim 35, wherein the cancer is breast cancer or prostate cancer.


SEQUENCE LISTING (23 sequences)

SEQ ID NO: 1 - protein, 15 aa, Homo sapiens
QPAASGDSDDDEPPP

SEQ ID NO: 2 - protein, 15 aa, Homo sapiens
DSDDDEPPPLPRLKR

SEQ ID NO: 3 - protein, 15 aa, Homo sapiens
KGRKFYGPEGPYGVF

SEQ ID NO: 4 - protein, 15 aa, Homo sapiens
FYGPEGPYGVFAGRD

SEQ ID NO: 5 - protein, 15 aa, Homo sapiens
KEALKDEYDDLSDLT

SEQ ID NO: 6 - protein, 15 aa, Homo sapiens
SDWESQFTFKYHHVG

SEQ ID NO: 7 - protein, 15 aa, Homo sapiens
EGEEPTVYSDEEEPK

SEQ ID NO: 8 - protein, 15 aa, Homo sapiens
GEEPTVYSDEEEPKD

SEQ ID NO: 9 - protein, 194 aa, Homo sapiens
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 10 - protein, 98 aa, Arabidopsis thaliana
EFTAEQLSQYNGTDESKPIYVAIKGRVFDVTTGKSFYGSGGDYSMFAGKDASRALGKMSKNEEDVSPSLEGLTEKEINTLNDWETKFEAKYPVVGRVV

SEQ ID NO: 11 - DNA, 6047 bp, Artificial Sequence
  Description of Artificial Sequence Synthetic polynucleotide
aaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattctagatacccatacgatgttcctgactatgcgggctatccctatgacgtcccggactatgcaggatcctatccatatgacgttccagattacgcttctagatgaactagagggcccgttt

SEQ ID NO: 12 - protein, 229 aa, Homo sapiens
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKNDSRYPYDVPDYAGYPYDVPDYAGSYPPYDVPDYASR

SEQ ID NO: 13 - protein, 229 aa, Artificial Sequence; identical to SEQ ID NO: 12
  Description of Artificial Sequence Synthetic polypeptide

SEQ ID NO: 14 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDADDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKNDSRYPYDVPDYAGYPYDVPDYAGSYPPYDVPDYASR

SEQ ID NO: 15 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYADEEEPKDESARKNDSRYPYDVPDYAGYPYDVPDYAGSYPPYDVPDYASR

SEQ ID NO: 16 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDADDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYADEEEPKDESARKNDSRYPYDVPDYAGYPYDVPDYAGSYPPYDVPDYASR

SEQ ID NO: 17 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDADDDEPPPLPRLKRRDFTPAELRRFDGVQDPRILMAINGKVFDVTKGRKFYGPEGPYGVFAGRDASRGLATFSLDKEALKDEYDDLSDLTAAQQETLSDWESQFTFKYHHVGKLLKEGEEPTVYADEEEPKDESARKNDSRYPYDVPDYAGYPYDVPDYAGSYPPYDVPDYASR

SEQ ID NO: 18 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEFDDLSDLTAAQQETLSDWESQFTFKYHHVGKLLKEGEEPTVYSDEEEPKDESARKNDSRYPYDVPDYAGYPYDVPDYAGSYPPYDVPDYASR

SEQ ID NO: 19 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVFSDEEEPKDESARKNDSRYPYDVPDYAGYPYDVPDYAGSYPPYDVPDYASR

SEQ ID NO: 20 - protein, 229 aa, Artificial Sequence
  Description of Artificial Sequence Synthetic polypeptide
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVFADEEEPKDESARKNDSRYPYDVPDYAGYPYDVPDYAGSYPPYDVPDYASR

SEQ ID NO: 21 - protein, 229 aa, Artificial Sequence
  Description of Artificial Sequence Synthetic polypeptide
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKNDSRYPYDVPDYAGYPYDVPDYAGSYPYDVPDYASR

SEQ ID NO: 22 - protein, 5 aa, Artificial Sequence
  Description of Artificial Sequence Synthetic peptide
TRGPV

SEQ ID NO: 23 - protein, 223 aa, Homo sapiens
MAAGDGDVKLGTLGSGSESSNDGGSESPGDAGAAAEGGGWAAAALALLTGGGEMLLNVALVALVLLGAYRLWVRWGRRGLGAGAGAGEESPATSLPRMKKRDFSLEQLRQYDGSRNPRILLAVNGKVFDVTKGSKFYGPAGPYGIFAGRDASRGLATFCLDKDALRDEYDDLSDLNAVQMESVREWEMQFKEKYDYVGRLLKPGEEPSEYTDEEDTKDHNKQD